(19) United States
(12) Patent Application Publication
(10) Pub. No.: US 2026/0257983 A1
(43) Pub. Date: Sep. 3, 2026

(54) COMPOSITIONS OF AND METHODS OF MAKING ULTRA-PURE TRICAPRILIN

(71) Applicant: CERECIN AUSTRALIA PTY LIMITED, Melbourne (AU)

(72) Inventor: Aikun Julie LIU, Geelong (AU)

(21) Appl. No.: 19/491,247

(22) PCT Filed: Jun. 10, 2024
(86) PCT No.: PCT/IB2024/055663

§ 371 Date: Dec. 8, 2025

Related U.S. Application Data
(60) Provisional application No. 63/472,229, filed on Jun. 9, 2023.
(60) Provisional application No. 63/607,779, filed on Dec. 8, 2023.

(51) Int. Cl. C07C 67/52; C07C 67/08; C07C 67/54; C07C 67/56; C07C 67/58; C07C 69/34

(52) U.S. Cl. CPC C07C 67/52; C07C 67/08; C07C 67/54; C07C 67/56; C07C 67/58; C07C 69/34

(57) ABSTRACT

The present disclosure provides methods of producing ultra-pure tricaprilin and compositions comprising the ultra-pure tricaprilin. The disclosure also provides methods of using the compositions for the treatment of diseases.

### CROSS REFERENCE TO RELATED APPLICATIONS

This application claims the benefit of the U.S. provisional application No. 63/472,229, filed Jun. 9, 2023 and 63/607,779, filed Dec. 8, 2023, the disclosures of which are each herein incorporated by reference in their entirety.

### BACKGROUND

### 1. Field

This invention generally relates to compositions of and methods of making ultra-pure tricaprilin.

### 2. Discussion of Related Art

Tricaprilin (which is synonymously called tricaprylin, trioctanoylglyceride, tricaprylyl glycerol, glycerol trioctanoin, tricapryloyl glycerol, octanoic acid-1,2,3-propanetriyl-ester, Glycerol tricaprylate, tricaprylyl Glycerol, glycerol tricaprylate, glycerol trioctanoate, glyceryl tricaprylate, caprylic triglyceride, tricaprilin, tricaprylyl glycerol etc.) is the triglyceride of caprylic acid which is usually manufactured by esterification of caprylic acid with Glycerol (glycerol).

Tricaprilin is used in pharmaceutical preparations, for example as a neutral carrier or excipient, as an absorption promoter and as a solubilizer for active drugs. It is also used as an oily phase to prepare water-in-oil-in-water multiple emulsions for incorporating water-soluble drugs and also for obtaining stable microcapsules. Being readily miscible with natural oils and surfactants, tricaprilin is used as fat component in two-phase foam baths, and in products like sunscreen creams, oils, fixative for perfumes and fragrances. High dose tricaprilin has also been used as active ingredients in pharmaceutical compositions for Alzheimer's diseases.

However, conventional production methods for manufacturing fatty acid polyol esters, particularly fatty acid glycerol esters, such as medium-chain triglycerides (MCTs), especially tricaprilin but also others, lead to relatively high degrees of impurities and by-products. These impurities are known to be toxic, especially toxic halide-based by-products and impurities. In particular, presence of these impurities and toxic by-products is not conducive to high dosage use. There is therefore an un-met need for production methods for ultra-pure tricaprilin, which may be broadly applicable to all MCTs.

### SUMMARY

In an aspect, the current disclosure encompasses an ultra-pure composition comprising at least about 99.2% by weight tricaprilin and about 0.8% by weight or less of impurities, wherein the tricaprilin is a tri-ester tricaprilin; and wherein the impurities comprise one or more of 2-monochloropropanediol, 2-monochloropropanediol monoester, 2-monochloropropanediol diester, 3-monochloropropanediol, 3-monochloropropanediol monoester, 3-monochloropropanediol diester, or any combination thereof. In an aspect, the ultra-pure composition comprising at least about 99.5% by weight tricaprilin and about 0.5% by weight or less of impurities. In an aspect, the ultra-pure composition comprising at least about 99.7% by weight tricaprilin and about 0.3% by weight or less of impurities. In an aspect, the ultra-pure composition may comprise 3-monochloropropanediol, 3-monochloropropanediol monoester, and 3-monochloropropanediol diester in a concentration of 35 ppb or less. In an aspect, the ultra-pure composition may comprise less than 35 ppb of each of 3-monochloropropanediol and 3-monochloropropanediol diester. In an aspect, the impurities may further comprise glycidol, mono-ester tricaprilin, di-ester tricaprilin, or tetra-ester tricaprilin. In an aspect, the impurities may further comprise unreacted reactants.

In an aspect, the current disclosure also encompasses a method of making an ultra-pure composition of tricaprilin, the method comprising: (a) feeding caprylic acid, a concentrated acid, glycerol, and water into a first reactor; (b) reacting caprylic acid, the concentrated acid, glycerol, and water to form a product mixture comprising tricaprilin and impurities in the first reactor, wherein the impurities comprise unreacted reactants and optionally undesired side products; (c) adding an organic solvent and an aqueous solution of a basic salt to a second reactor forming a biphasic mixture comprising a top organic phase and a bottom aqueous phase; (d) adding portions of the product mixture from step (b) into the biphasic mixture in step (c) to remove the unreacted reactants, wherein the unreacted reactants are in the aqueous phase; (e) removing the bottom aqueous phase from the second reactor; (f) treating the top organic phase with charcoal; (g) distilling off the organic solvent and leaving tricaprilin and impurities in the second reactor; and (h) recrystallizing the tricaprilin and impurities to remove the impurities and form the ultra-pure tricaprilin. In some aspects, step (a) may further comprise: (ai) co-feeding caprylic acid and concentrated acid into the first reactor to form a mixture; (aii) heating the mixture to a first temperature under an inert atmosphere; and (aiii) adding Glycerol and water to the mixture over a first specified time. In some aspects, the step (b) may further comprise: (bi) stirring the product mixture for a second specified time at a second temperature; (bii) removing a first in process sample from the first reactor; (biii) checking the first in process sample for conversion, wherein conversion is reaction of hydroxyl groups in the glycerol to ester groups in tricaprilin; and (biv) repeating step (bii) to step (biii) until conversion occurs. In some aspects, the step (g) may further comprise (gi) removing a second in process sample from the second reactor; (gii) measuring amount of the organic solvent remaining in the tricaprilin and impurities; and (giii) repeating steps (gi) to (gii) until the amount of the organic solvent remaining is 500 ppm or less. In some aspects, the step (h) may further comprise: (hi) adding a polar solvent to the tricaprilin and impurities forming a second mixture comprising a polar solvent, tricaprilin, and impurities; (hii) cooling the second mixture to a third temperature; (hiii) adding seeding crystals to the second mixture forming a third mixture comprising seeding crystals and the second mixture; (hiv) stirring the third mixture for a third specified time at a third temperature; (hv) cooling the third mixture to a fourth temperature in a fourth specified time; (hvi) maintaining the third mixture at the fourth temperature for a fifth specified time; (hvii) adding water to the third mixture; (hviii) maintaining the third mixture at the fourth temperature; (hix) filtering the third mixture on a filter cooled at the fourth temperature; (hx) washing the third mixture with an aqueous solution of the polar solvent at a fifth temperature; (hxi) repeating the washing step (hx); (hxii) collecting crystals of tricaprilin; and (hxiii) evaporating residual solvent from the crystals using a rotary evaporator. In some aspects, the concentrated acid comprises sulfuric acid. In some aspects of the method disclosed herein, the first temperature ranges from about 90° C. to about 120° C. In some aspects, the first specified time ranges from about 3 hours to about 5 hours. In some aspects, the second temperature ranges from about 90° C. to about 120° C. In some aspects, the second specified time ranges from about 15 hours to about 24 hours.

In an aspect, the current disclosure also encompasses a pharmaceutical composition comprising the ultra-pure composition provided herein. Also, disclosed herein is a method of prophylactically or therapeutically treating a disease or a disorder in a subject in need thereof, the method comprising administering a composition comprising ultra-pure tricaprilin to the subject. Non-limiting examples of diseases or disorders craniocerebral trauma, stroke, hypoxia, myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, chronic inflammatory bowel diseases, ulcerative colitis, Crohn's disease, lyosomal storage diseases, sphingolipidosis, especially Niemann-Pick disease, diabetes mellitus, migraine and migraine headaches, epilepsy, especially infantile spasms as well as effects or side-effects of chemotherapy. In an aspect, the ultra-pure composition of tricaprilin is administered orally.

### BRIEF DESCRIPTION OF THE FIGURES

FIG. 1 shows a schematic of the reaction resulting in generation of monochloropropanediol esters during production of tricaprilin.

FIG. 2 shows a schematic of the reaction scheme for the production of tricaprilin.

FIG. 3 shows a general process of making an ultra-pure tricaprilin composition, in accordance with a representative aspect of the present invention.

FIG. 4 shows a general process of making an ultra-pure tricaprilin composition, in accordance with a representative aspect of the present invention.

FIG. 5 provides a schematic for production of tricaprilin (TARI-01).

FIG. 6 shows the technical set up for a lab scale experiment. The reactor was equipped (from left to right) with a N₂ Inlet, a mechanical stirred, a thermocouple and a Dean Stark receiver.

FIG. 7 shows a lab scale apparatus for the recrystallization of tricaprilin.

FIG. 8A shows a CG-MS assay for glycidol for Exp23 AA8219-3.

FIG. 8B shows a CG-MS assay for 3-monochloropropanediol (3-MCPD) and 2-monochloropropanediol (2-MCPD) for Exp23 AA8219-3.

FIG. 8C shows a CG-MS assay for glycidol for Exp23 AA5433-1.

FIG. 8D shows a CG-MS assay for 3-MCPD and 2-MCPD for Exp23 AA5433-1.

FIG. 9 shows a manufacturing flow chart for a process of manufacturing an ultra-pure tricaprilin composition, in accordance with a representative aspect of the present invention.

### DETAILED DESCRIPTION

The following description of the aspects of the invention is not intended to limit the invention to these aspects but rather to enable a person skilled in the art to make and use this invention.

In an aspect, the current disclosure is based on extensive experimentation to develop a method for producing ultra-pure tricaprilin. These experiments provided several unexpected results and insights and led to the development of a novel method for production of tricaprilin with minimal impurities.

Conventional production methods for manufacturing fatty acid polyol esters, particularly fatty acid glycerol esters, such as medium-chain triglycerides (MCTs), especially tricaprilin but also others, including the subsequent multistage refinement treatment (above multistage distillation at elevated temperatures), lead to relatively high degrees of impurities and by-products particularly due to high process temperatures needed and the used starting materials and reagents, particularly halogen-containing substances (e.g., originating from hydrochloric acid HCl or metal-based catalysts used in the esterification process). Especially, when triglycerides with low acid and hydroxyl values and high esterification degrees are required, the mandatory drastic esterification conditions, especially high temperatures over 180° C. and up to 230° C., the necessary subsequent multistage refinement treatment (above multistage distillation at elevated temperatures) and the use of metal-based catalysts lead to the formation of high contents of toxic by-products and impurities, especially toxic halide-based by-products and impurities.

Above all, conventional production methods for manufacturing fatty acid polyol esters, particularly fatty acid glycerol esters, including the subsequent multistage refinement treatment (above multistage distillation at elevated temperatures), usually produce relatively high levels of impurities and by-products, especially toxic impurities and by products, such as, genotoxic glycidol esters of fatty acids (synonymously also called glycidyl esters of fatty acids or simply GEs of fatty acids or only GEs) and nephrotoxic fatty acid esters of monochloropropanediols (synonymously called MCPD fatty acid esters or simply MCPD esters), especially fatty acid esters of 3-monochloropropanediol (3-MCPD fatty acid esters or 3-MCPD esters) and fatty acid esters of 2-monochloropropanediol (2-MCPD fatty acid esters or 2-MCPD esters).

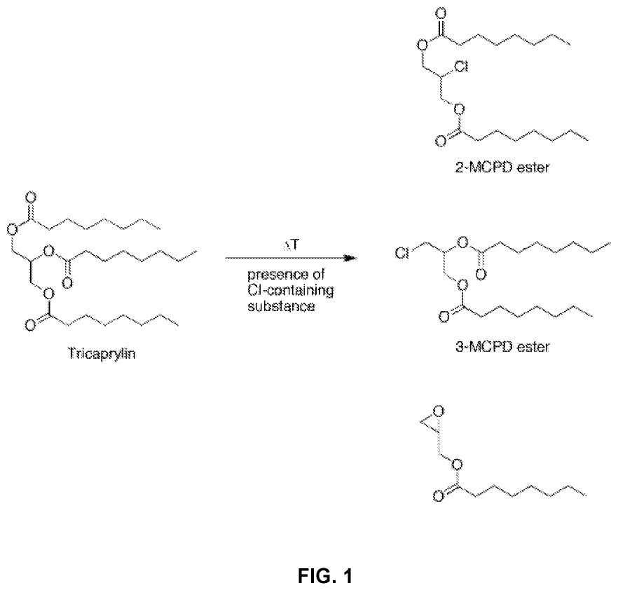

The exemplary reaction scheme of FIG. 1 illustrates the formation of the three aforenamed toxic by-products, i.e., 3-MCPD fatty acid ester, 2-MCPD fatty acid ester and glycidol fatty acid ester for the triglyceride tricaprilin (selected as a non-limiting example), whose formation occurs when high temperatures (e.g., temperatures of 200° C. or more) are applied to the fatty acid triglyceride (e.g., tricaprilin) in the presence of chlorine-containing substances originating from the production process. The substance 3-chloro-1,2-propanediol originates from glycerol when reacting to 3-MCPD in the presence of chlorine-containing substances or chlorides, respectively (see FIG. 1).

Above all with respect to pharmaceutical and nutritional applications, controlling the impurity profile is crucial and compulsory, especially for known toxic by-products such as the three aforenamed toxic by-products, i.e., 3-MCPD fatty acid ester, 2-MCPD fatty acid ester and glycidol fatty acid ester.

Although it has been well known for decades that, due to the harsh process conditions occurring during the conventional esterification/condensation methods of fats and oils, undesired and toxic by-products are formed, no attempts have been made to avoid such harsh conditions by improving or modifying the production processes. Mild esterification conditions using biobased catalysts and lower esterification temperatures reduce the formation of toxic by-products, such as the three aforenamed toxic by-products (i.e., 3-MCPD fatty acid ester, 2-MCPD fatty acid ester and glycidol fatty acid ester), but at the same time only lead to triglycerides with undesirably high acid and hydroxyl values and relatively low triglyceride contents at low conversion rates and yields.

Consequently, instead of trying to avoid the formation of undesired and toxic by-products, rather cost-intensive and burdensome pre- and/or post-treatment methods are affected in order to remove these undesired and toxic by-products from the desired final product obtained under harsh esterification conditions (i.e., especially high temperatures over 180° C. and up to 230° C. and use of metal-based catalysts, etc.). Such burdensome post treatment methods comprise, inter alia, e.g., the addition of strongly alkaline reagents such as KOH, multistage distillation cascades, heat-treatment with strongly alkaline aqueous solutions etc. (as described in, for example, WO 2019/038320 A1 and WO 2014/012548 A1 which are each incorporated herein by reference in their entirety). These post-treatment methods also affect and deteriorate the product quality as such and overall yields and efficiency. For instance, WO 2021/070209 A1, incorporated herein by reference in its entirety, discloses a complex multistage extraction process for the purification of triglycerides, using a saline/organic solvent two-phase extraction system also with chlorinated organic solvents.

However, these post-treatment methods mostly fail to remove last traces of these undesired and toxic by-products, and these traces then remain in the final product. This is of particular danger when applying high dosages of the fatty acid polyol esters, particularly fatty acid glycerol esters, e.g., MCTs such as tricaprilin, in pharmaceutical and nutritional applications (above all in case of parenteral application), which leads to an undesired accumulation of such traces of undesired and toxic by-products in the human body, thus also exceeding the authorized threshold levels of the undesired and toxic by-products as regulated by law. Moreover, such cost-intensive and burdensome post-treatment methods which aim to effect a only subsequent or retroactive removal of the undesired and toxic by-products are also inefficient since these post-treatment methods often lead to an undesired entrainment of product and do also not increase or influence product quality or product purity such as the esterification degree or the residual content of other impurities (such as, e.g., the total halogenated substance content or the total acid content).

It would be desirable to conceive efficient production processes which directly produce high-grade fatty acid polyol esters, particularly fatty acid glycerol esters, such as MCTs (e.g., tricaprilin), having high degrees of purity or minimum levels of by-products and impurities, respectively, as well as exhibiting at the same time high esterification degrees and good stabilities, especially storage-stabilities, and high active ingredient contents, especially without the necessity of additional cost-intensive and burdensome post-treatment operations. Consequently, the prior art has not lacked attempts to find efficient production processes but failed to improve the efficiency and performance of existing processes. But so far, however, no efficient production processes have been conceived in the prior art. Also, access to such high-grade fatty acid polyol esters, particularly fatty acid glycerol esters, such as MCTs (e.g., tricaprilin), is not or not easily possible according to the prior art.

The problem underlying the present invention is thus providing an efficient process for producing triglycerides of fatty acids (i.e., fatty acid glycerol triesters), above all tricaprilin, especially with a high purity degree, wherein the aforementioned disadvantages and/or drawbacks of the prior art should be at least partially avoided or even at least essentially overcome.

Such production process should especially make the respective fatty acid polyol esters, particularly fatty acid glycerol esters, such as MCTs (above all tricaprilin), accessible in an efficient way, especially with improved qualities and without significant amounts of toxic by-products or impurities, which production process should be feasible on an industrial level, especially on a large-scale level, and without excessive and cost-intensive post-treatment as envisaged in the prior art processes.

Accordingly, the disclosure herein provides an ultra-pure composition comprising at least about 99.2% by weight tricaprilin and about 0.8% by weight or less of impurities as well as methods of preparing an ultra-pure composition comprising at least about 99.2% by weight tricaprilin and about 0.8% or less by weight of impurities. In an aspect, the disclosure herein provides an ultra-pure composition comprising at least about 99.5% by weight tricaprilin and about 0.5% by weight or less of impurities as well as methods of preparing an ultra-pure composition comprising at least about 99.5% by weight tricaprilin and about 0.5% or less by weight of impurities.

### Definitions

The terms “a” and “an” and “the” and similar referents as used herein refer to both the singular and the plural, unless otherwise indicated herein or clearly contradicted by context.

The term “about” as used herein refers to greater or lesser than the value or range of values stated by 1/10 of the stated values but is not intended to limit any value or range of values to only this broader definition. For instance, a value of “about 30%” means a value of between 27% and 33%. Each value or range of values preceded by the term “about” is also intended to encompass the aspect of the stated absolute value or range of values.

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Moreover, the present disclosure also contemplates that in some aspects, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Pharmaceutical composition” means a mixture of substances suitable for administering to an individual that includes a pharmaceutical agent. As used herein a pharmaceutical composition comprises one or more of the compounds as disclosed herein compounded with suitable pharmaceutical excipients.

As used herein, the term “patient”, “subject”, including “test subject” refers to any organism to which provided compound or compounds described herein are administered in accordance with the present disclosure e.g., for experimental, diagnostic, prophylactic, and/or therapeutic purposes. Typical subjects include animals (e.g., mammals such as mice, rats, rabbits, non-human primates, humans, insects, worms etc.). In an aspect, a subject is a human. In some aspects, a subject may be suffering from a tumor and/or cancer as disclosed herein. In some aspects, the cancer is a leukemia. In some aspects, the cancer is colorectal cancer.

The term “effective amount” as used herein is defined as the amount of the molecules of the present disclosure that are necessary to result in the desired physiological change in the cell or tissue to which it is administered. The term “therapeutically effective amount” as used herein is defined as the amount of the molecules or compositions of the present disclosure that achieves a desired effect with respect to cancer. In this context, a “desired effect” is synonymous with “an antitumor activity” or “an anti-cancer activity”. A skilled artisan readily recognizes that in many cases the molecules may not provide a cure but may provide a partial benefit, such as alleviation or improvement of at least one symptom or parameter. In some aspects, a physiological change having some benefit is also considered therapeutically beneficial. Thus, in some aspects, an amount of molecules that provides a physiological change is considered an “effective amount” or a “therapeutically effective amount.”

The term “ultra-pure” as used herein refers to tricaprilin that comprises at least about 99.2% by weight tricaprilin. Ultra-pure active pharmaceutical ingredient (API) contains the highest purity with lowest related substances including lowest level of genotoxic impurities compared to any currently available MCT oils and other sources of tricaprilin. In an aspect, the tricaprilin comprises less than 0.8% by weight of impurities. The term “impurities” as used herein and unless otherwise specified herein, refers to undesired compounds and elements, such as, for example, unreacted reactants and undesired side and/or by-products. In some instances, the impurity(s) may have negative and/or deleterious effects on a subject; especially when the subject is exposed to amounts of the impurity(s) that exceed the authorized threshold levels; either in a single instance or over time. In an aspect, the ultra-pure tricaprilin disclosed herein is suitable for administration to a subject at a high dose of at least about 20 mg/day. In an aspect, the ultra-pure tricaprilin is suitable for administration to a subject at a high dose of at least about 30 mg/day. In an aspect, the ultra-pure tricaprilin is suitable for administration to a subject at a high dose of at least about 40 mg/day. The per day amount may be split into multiple doses or be administered as a single dose.

### I. Compositions

In an aspect, the current disclosure encompasses an ultra-pure composition comprising at least about 99.2% by weight tricaprilin and about 0.8% by weight or less of impurities, wherein the tricaprilin is a tri-ester tricaprilin. In an aspect, the ultra-pure composition of the present disclosure may include at least about 99.3% by weight tricaprilin and about 0.7% by weight or less of impurities. In some aspects, the ultra-pure composition may include at least about 99.4% by weight tricaprilin and about 0.6% by weight or less of impurities. In an aspect, the ultra-pure composition of the present disclosure may include at least about 99.5% by weight tricaprilin and about 0.5% by weight or less of impurities. In some aspects, the ultra-pure composition may include at least about 99.6% by weight tricaprilin and about 0.4% by weight or less of impurities. In some aspects, the ultra-pure composition may include at least about 99.7% by weight tricaprilin and about 0.3% by weight or less of impurities. In some aspects, the ultra-pure composition may include at least about 99.8% by weight tricaprilin and about 0.2% by weight or less of impurities. In some aspects, the ultra-pure composition may include at least about 99.9% by weight tricaprilin and about 0.1% by weight or less of impurities. In some aspects, the ultra-pure composition may include at least about 99.99% by weight tricaprilin and about 0.01% by weight or less of impurities.

In some aspects, the impurities may comprise any one or more of 2-monochloropropanediol, 2-monochloropropanediol monoester, 2-monochloropropanediol diester, 3-monochloropropanediol, 3-monochloropropanediol monoester, 3-monochloropropanediol diester, or any combination thereof. In some aspects, the impurities may include unreacted reactants and optionally undesired side products. In some aspects, the impurities may include 2-monochloropropanediol, 2-monochloropropanediol monoester, 2-monochloropropanediol diester, 3-monochloropropanediol, 3-monochloropropanediol monoester, 3-monochloropropanediol diester, may include unreacted reactants and optionally undesired side products, or any combination thereof.

In some aspects, 3-monochloropropanediol, 3-monochloropropanediol monoester, and 3-monochloropropanediol diester may be present in a concentration of 50 ppb or less. In some aspects, 3-monochloropropanediol, 3-monochloropropanediol monoester, and 3-monochloropropanediol diester may be present in a concentration of 45 ppb or less. In some aspects, 3-monochloropropanediol, 3-monochloropropanediol monoester, and 3-monochloropropanediol diester may be present in a concentration of 40 ppb or less. In some aspects, 3-monochloropropanediol, 3-monochloropropanediol monoester, and 3-monochloropropanediol diester may be present in a concentration of 35 ppb or less. In some aspects, 3-monochloropropanediol, 3-monochloropropanediol monoester, and 3-monochloropropanediol diester may be present in a concentration of 30 ppb or less. In some aspects, 3-monochloropropanediol, 3-monochloropropanediol monoester, and 3-monochloropropanediol diester may be present in a concentration of 25 ppb or less. In some aspects, 3-monochloropropanediol, 3-monochloropropanediol monoester, and 3-monochloropropanediol diester may be present in a concentration of 20 ppb or less. In some aspects, 3-monochloropropanediol, 3-monochloropropanediol monoester, and 3-monochloropropanediol diester may be present in a concentration of 15 ppb or less. In some aspects, 3-monochloropropanediol, 3-monochloropropanediol monoester, and 3-monochloropropanediol diester may be present in a concentration of 14 ppb or less. In some aspects, 3-monochloropropanediol, 3-monochloropropanediol monoester, and 3-monochloropropanediol diester may be present in a concentration of 13 ppb or less. In some aspects, 3-monochloropropanediol, 3-monochloropropanediol monoester, and 3-monochloropropanediol diester may be present in a concentration of 12 ppb or less. In some aspects, 3-monochloropropanediol, 3-monochloropropanediol monoester, and 3-monochloropropanediol diester may be present in a concentration of 11 ppb or less. In some aspects, 3-monochloropropanediol, 3-monochloropropanediol monoester, and 3 monochloropropanediol diester may be present in a concentration of 10 ppb or less. In some aspects, 3-monochloropropanediol, 3-monochloropropanediol monoester, and 3-monochloropropanediol diester may be present in a concentration of 9 ppb or less. In some aspects, 3-monochloropropanediol, 3-monochloropropanediol monoester, and 3-monochloropropanediol diester may be present in a concentration of 8 ppb or less. In some aspects, 3-monochloropropanediol, 3-monochloropropanediol monoester, and 3-monochloropropanediol diester may be present in a concentration of 7 ppb or less. In some aspects, 3-monochloropropanediol, 3-monochloropropanediol monoester, and 3 monochloropropanediol diester may be present in a concentration of 6 ppb or less. In some aspects, 3-monochloropropanediol, 3-monochloropropanediol monoester, and 3-monochloropropanediol diester may be present in a concentration of 5 ppb or less. In some aspects, 3-monochloropropanediol, 3-monochloropropanediol monoester, and 3-monochloropropanediol diester may be present in a concentration of 4 ppb or less. In some aspects, 3-monochloropropanediol, 3-monochloropropanediol monoester, and 3-monochloropropanediol diester may be present in a concentration of 3 ppb or less. In some aspects, 3-monochloropropanediol, 3-monochloropropanediol monoester, and 3-monochloropropanediol diester may be present in a concentration of 2 ppb or less. In some aspects, 3-monochloropropanediol, 3-monochloropropanediol monoester, and 3-monochloropropanediol diester may be present in a concentration of 1 ppb or less.

In some aspects, 3-monochloropropanediol, 3-monochloropropanediol monoester, and 3-monochloropropanediol diester may be present in a concentration of 35 ppb or less.

In some aspects, 3-monochloropropanediol, 3-monochloropropanediol monoester, and 3-monochloropropanediol diester may be present in a concentration of 15 ppb or less.

In some aspects, of each of 3-monochloropropanediol and 3-monochloropropanediol diester may be present in a concentration of 50 ppb or less. In some aspects, of each of 3-monochloropropanediol and 3-monochloropropanediol diester may be present in a concentration of 45 ppb or less. In some aspects, of each of 3-monochloropropanediol and 3-monochloropropanediol diester may be present in a concentration of 40 ppb or less. In some aspects, of each of 3-monochloropropanediol and 3-monochloropropanediol diester may be present in a concentration of 35 ppb or less. In some aspects, of each of 3-monochloropropanediol and 3-monochloropropanediol diester may be present in a concentration of 30 ppb or less. In some aspects, of each of 3-monochloropropanediol and 3-monochloropropanediol diester may be present in a concentration of 25 ppb or less. In some aspects, of each of 3-monochloropropanediol and 3-monochloropropanediol diester may be present in a concentration of 15 ppb or less. In some aspects, of each of 3-monochloropropanediol and 3-monochloropropanediol diester may be present in a concentration of 14 ppb or less. In some aspects, of each of 3-monochloropropanediol and 3-monochloropropanediol diester may be present in a concentration of 13 ppb or less. In some aspects, of each of 3-monochloropropanediol and 3-monochloropropanediol diester may be present in a concentration of 12 ppb or less. In some aspects, of each of 3-monochloropropanediol and 3-monochloropropanediol diester may be present in a concentration of 11 ppb or less. In some aspects, of each of 3-monochloropropanediol and 3-monochloropropanediol diester may be present in a concentration of 10 ppb or less. In some aspects, of each of 3-monochloropropanediol and 3-monochloropropanediol diester may be present in a concentration of 9 ppb or less. In some aspects, of each of 3-monochloropropanediol and 3-monochloropropanediol diester may be present in a concentration of 8 ppb or less. In some aspects, of each of 3-monochloropropanediol and 3-monochloropropanediol diester may be present in a concentration of 7 ppb or less. In some aspects, of each of 3-monochloropropanediol and 3-monochloropropanediol diester may be present in a concentration of 6 ppb or less. In some aspects, of each of 3-monochloropropanediol and 3-monochloropropanediol diester may be present in a concentration of 5 ppb or less. In some aspects, of each of 3-monochloropropanediol and 3-monochloropropanediol diester may be present in a concentration of 4 ppb or less. In some aspects, of each of 3-monochloropropanediol and 3-monochloropropanediol diester may be present in a concentration of 3 ppb or less. In some aspects, of each of 3-monochloropropanediol and 3-monochloropropanediol diester may be present in a concentration of 2 ppb or less. In some aspects, of each of 3-monochloropropanediol and 3-monochloropropanediol diester may be present in a concentration of 1 ppb or less.

In some aspects, of each of 3-monochloropropanediol and 3-monochloropropanediol diester may be present in a concentration of 35 ppb or less.

In some aspects, of each of 3-monochloropropanediol and 3-monochloropropanediol diester may be present in a concentration of 15 ppb or less.

In some aspects, the impurities may include glycidol, mono-ester tricaprilin, di-ester tricaprilin, or tetra-ester tricaprilin. In some aspects, the impurities may include glycidol, mono-ester tricaprilin, di-ester tricaprilin, or tetra-ester tricaprilin. In an aspect, glycidol impurities may be present at 50 ppb or less. In an aspect, glycidol impurities may be present at 40 ppb or less. In an aspect, glycidol impurities may be present at 35 ppb or less. In an aspect, glycidol impurities may be present at 30 ppb or less. In an aspect, glycidol impurities may be present at 20 ppb or less.

In an aspect, the ultra-pure tricaprilin conforms to the limits as provided in Table 1 below.

TABLE 1
Characteristics and impurity limits on an exemplary
ultra-pure tricaprilin composition

| | |
|---|---|
| Parameter | Limit |

| | |
|---|---|
| Appearance | clear, colorless or |
| | slightly yellowish liquid |
| Color | max. 100 APHA |
| Identity (FT-IR) | conforms with |
| | reference spectrum |
| Identity (GC-FID) | retention time conforms |
| Assay (GC-FID) | 97.0-103.0% (w/w) |
| Purity | Not less than 99.2% |

Related Substances (GC-FID, % area)

| Glyceryl-monoheptanoate dioctanoate | NMT 0.5% | |
|---|---|---|
| Any unspecified related substance | max. 0.2% | |
| Total related substances | max. 0.8% | |
| Acid Value | max. 0.2 | mg KOH/g |
| Residue on Ignition (Sulphated Ash) | max. 0.1% | |
| Elemental impurities (ICP-MS) | | |
| Cd | 0.08 | ppm |
| Pb | 0.08 | ppm |
| As | 0.25 | ppm |
| Hg | 0.50 | ppm |
| Co | 1 | ppm |
| V | 2 | ppm |
| Ni | 3 | ppm |
| Water (K.F.) | max. 0.2% | |
| Residual solvents | | |
| Methyl tert-butyl ether (MTBE) | max. 500 | ppm |
| Methanol | max. 300 | ppm |
| Microbiology | | |
| Total Aerobic Microbial Count | ≤100 | CFU/g |
| Total Yeasts and Molds Count | ≤10 | CFU/g |
| E. coli | absent in 1 g | |
| MCPD and Glycidol (GC-MS) | | |
| Glycidyl equivalent | max. 35 | ppb |
| 2-MCPD equivalent | max. 35 | ppb |
| 3-MCPD equivalent | max. 35 | ppb |

In some aspects the impurities may be measured by suitable analytical techniques. Suitable analytical techniques include but are not limited to gas chromatography, mass spectrometry, or chemical analysis.

In some aspects, the ultra-pure composition of tricaprilin may be further characterized by measuring any one of or a combination of acid value, saponification value, refractive index, water content, total aerobic microbial count, total yeast and mold count, elemental impurities by ICP-MS.

### II. Method of Making Ultra-Pure Composition

The present disclosure further encompasses methods of making an ultra-pure composition of tricaprilin as disclosed herein. The general reaction is an esterification reaction in which an acid may react with an alcohol resulting in the formation of an ester.

The method generally includes reacting an alkyl carboxylic acid with an alkyl triol at a specified temperature to form a product mixture and purifying the product mixture to obtain the ultra-pure composition of tricaprilin. The product mixture includes tricaprilin and impurities. The purifying step includes separating the tricaprilin and impurities in an organic solvent, distilling the organic solvent, and recrystallizing the product mixture to obtain the ultra-pure composition of tricaprilin.

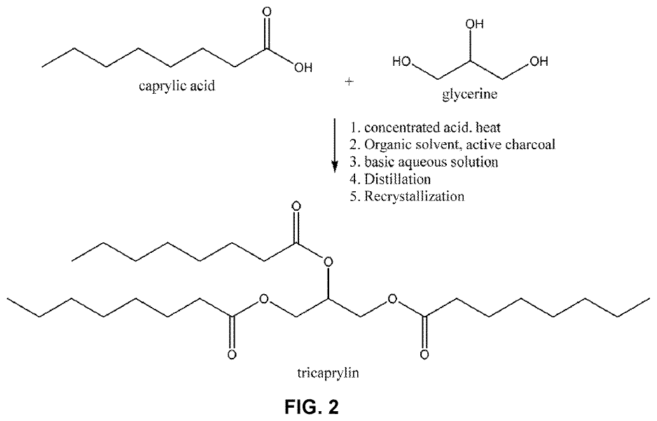

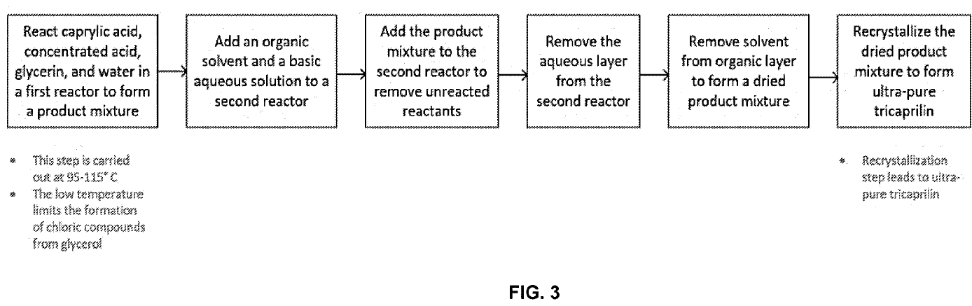

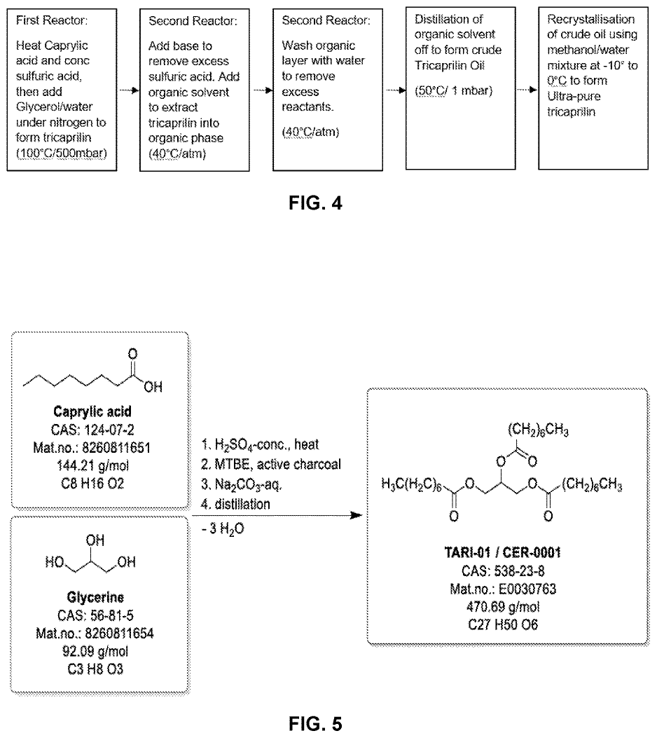

FIG. 2 shows the general reaction according to the present disclosure. The reacting step includes (a) feeding caprylic acid, a concentrated acid, glycerol, and water into a first vessel and (b) reacting caprylic acid, concentrated acid, glycerol, and water to form a product mixture comprising tricaprilin and impurities in a first vessel. The purification step generally includes (c) adding an organic solvent and an aqueous solution of a basic salt to a second vessel forming a biphasic mixture comprising a top organic phase and a bottom aqueous phase; (d) adding portions of the product mixture from step (b) into the biphasic mixture in step (c) to remove the unreacted reactants; (e) removing the bottom aqueous phase from the second vessel; (f) treating the top organic phase with charcoal; (g) distilling off the organic solvent and leaving tricaprilin and impurities in the second vessel; and (h) recrystallizing the tricaprilin and impurities to remove the impurities and form the ultra-pure tricaprilin. FIG. 3 illustrates the steps in the overall process. FIG. 4 further elaborates on which vessel each step may be conducted in.

In some aspects, the impurities in the product mixture may include unreacted reactants and optionally undesired side products. In some aspects, the unreacted reactants may be in the aqueous phase.

### The Reacting Step

In some aspects, the reacting step comprises (a) feeding caprylic acid, a concentrated acid, glycerol, and water into a first vessel and (b) reacting caprylic acid, concentrated acid, glycerol, and water to form a product mixture comprising tricaprilin and impurities in the first vessel. In some aspects, the feeding step may further comprise co-feeding caprylic acid and concentrated acid into the first vessel to form a mixture; heating the mixture to a first temperature under an inert atmosphere; and adding glycerol and water to the mixture.

In some aspects, the reacting step may further include stirring the product mixture at the first temperature for a specified time; removing a first in process sample from the first vessel; checking the first in process sample for conversion; and repeating the steps until conversion occurs.

In some aspects, the vessel may be an ordinary reactor, such as a tubular reactor or a tank reactor.

In an aspect, the caprylic acid used in the current method may be at least about 99% pure. In an aspect, the caprylic acid has a profile as provided below in Table 2.

TABLE 2
Exemplary profile of caprylic acid for use in the disclosed method.

| Parameter | Limit |
|---|---|
| Appearance | clear, colorless or slightly |
| | yellowish liquid |
| Identity (FT-IR) | practically identical with |
| | reference spectrum |
| Assay (Titration) | 99-101% |
| Purity (GC-FID, % area) | |
| Caprylic acid (C8:0) | min. 99.5% |
| Hexanoic acid (C6:0) | max. 0.5% |
| Heptanoic acid (C7:0) | max. 0.5% |
| Nonanoic acid (C9:0) | max. 0.5% |
| Decanoic acid (C10:0) | max. 0.5% |
| Dodecanoic acid (C12:0) | max. 0.5% |
| Each unspecified impurity | max. 0.3% |
| Residue on ignition, 1 g sample | max. 0.1% |

Similarly, the glycerol for use with the disclosed method may be of high purity, for example at least about 98% pure. In an exemplary aspect, the glycerol for use herein has a profile as provided in Table 3.

TABLE 3
Exemplary profile of glycerol for use in the disclosed method.

| Parameter | Limit |
|---|---|
| Appearance | clear liquid |
| Color (Appearance of solution | colorless |
| Identity (Refractive Index, nD₂0) | 1.470-1.475 |
| Identity (GC, Retention time) | retention time conforming |
| Water (KF, 2 g sample) | max. 2.0% |
| Assay (Titration, on anhydrous) | 98.0-101.0% |
| Chemical purity (GC, % norm.area) | |
| Diethylene glycol (Imp. A) | max. 0.1% |
| Ethylene glycol | max. 0.1% |
| Each unspecified impurity | max. 0.1% |
| Total impurities with retention | max. 0.5% |
| time greater than glycerol | |
| Chloride | max. 10 ppm |

The concentrated acid may be selected based on the dehydrating properties of the acid. The dehydrating property may be crucial in esterification reactions. The water produced as a by-product of the esterification reaction may be removed through dehydration, shifting the equilibrium of the esterification reaction towards the formation of the ester.

In some aspects, the acid may be concentrated sulfuric acid, phosphoric acid, or a combination thereof. In an aspect, the concentrated acid may be sulfuric acid.

In some aspects, the concentrated sulfuric acid may be between about 95 weight percent (wt %) and under about 99.99 wt %. For example, the concentrated sulfuric acid may be about 95.1 wt %, about 95.2 wt %, about 95.3 wt %, about 95.4 wt %, about 95.5 wt %, about 95.6 wt %, about 95.7 wt %, about 95.8 wt %, about 95.9 wt %, about 96.0 wt %, about 96.1 wt %, about 96.2 wt %, about 96.3 wt %, about 96.4 wt %, about 96.5 wt %, about 96.6 wt %, about 96.7 wt %, about 96.8 wt %, about 96.9 wt %, about 97.0 wt %, about 97.1 wt %, about 97.2 wt %, about 97.3 wt %, about 97.4 wt %, about 97.5 wt %, about 97.6 wt %, about 97.7 wt %, about 97.8 wt %, about 97.9 wt %, about 99.0 wt %, about 99.1 wt %, about 99.2 wt %, about 99.3 wt %, about 99.4 wt %, about 99.5 wt %, about 99.6 wt %, about 99.7 wt %, about 99.8 wt %, about 99.9 wt %, or about 99.99 wt %.

In some aspects, the first temperature may range from about 90° C. to about 120° C. For example, the first temperature may be about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., about 105° C., about 106° C., about 107° C., about 108° C., about 109° C., about 110° C., about 111° C., about 112° C., about 113° C., about 114° C., about 115° C., about 116° C., about 117° C., about 118° C., about 119° C., or about 120° C. In a preferred aspect, the first temperature may range from about 95° C. to about 115° C.

In some aspects, the inert atmosphere may be nitrogen, argon, or a mixture thereof.

In some aspects, the first vessel may be maintained at a pressure of between about 250 mbar to about 600 mbar. For example, the pressure may be about 260 mbar, about 270 mbar, about 280 mbar, about 290 mbar, about 300 mbar, about 310 mbar, about 320 mbar, about 330 mbar, about 340 mbar, about 350 mbar, about 360 mbar, about 370 mbar, about 380 mbar, about 390 mbar, about 400 mbar, about 410 mbar, about 420 mbar, about 430 mbar, about 440 mbar, about 450 mbar, about 460 mbar, about 470 mbar, about 480 mbar, about 490 mbar, about 500 mbar, about 510 mbar, about 520 mbar, about 530 mbar, about 540 mbar, about 550 mbar, about 560 mbar, about 570 mbar, about 580 mbar, about 590 mbar, or about 600 mbar.

In some aspects, the glycerol to water ratio may range from 2:1 to 1:2. For example, the ratio may be around 2:1, around 1.5:1, around 1:1, around 1:1.5, or around 1:2.

In some aspects, the specified time may range from about 2 hours to about 17 hours. In some aspects, the specified time may range from about 3 hours to about 16 hours. In some aspects, the specified time may range from about 4 hours to about 15 hours. In some aspects, the specified time may range from about 5 hours to about 14 hours. In some aspects, the specified time may range from about 6 hours to about 13 hours. In some aspects, the specified time may range from about 7 hours to about 12 hours. In some aspects, the specified time may range from about 8 hours to about 11 hours. In some aspects, the specified time may range from about 9 hours to about 10 hours. In some aspects the specified time may range from about 3 hours to about 5 hours.

The conversion occurs when the hydroxyl groups in the glycerol react to form ester groups in tricaprilin. After conversion is achieved, the product mixture may undergo purification to produce the ultra-pure composition of tricaprilin.

### The Purifying Step

In an aspect, the purification step may comprise one or more of (c) adding an organic solvent and an aqueous solution of a basic salt to a second vessel forming a biphasic mixture comprising a top organic phase and a bottom aqueous phase; (d) adding portions of the product mixture from step (b) into the biphasic mixture in step (c) to remove the unreacted reactants; (e) removing the bottom aqueous phase from the second vessel; (f) treating the top organic phase with charcoal; (g) distilling off the organic solvent and leaving tricaprilin and impurities in the second vessel; and (h) recrystallizing the tricaprilin and impurities to remove the impurities and form the ultra-pure tricaprilin. In an aspect, the purification step may comprise all of (c) adding an organic solvent and an aqueous solution of a basic salt to a second vessel forming a biphasic mixture comprising a top organic phase and a bottom aqueous phase; (d) adding portions of the product mixture from step (b) into the biphasic mixture in step (c) to remove the unreacted reactants; (e) removing the bottom aqueous phase from the second vessel; (f) treating the top organic phase with charcoal; (g) distilling off the organic solvent and leaving tricaprilin and impurities in the second vessel; and (h) recrystallizing the tricaprilin and impurities to remove the impurities and form the ultra-pure tricaprilin

In some aspects, the product mixture from the first vessel may be added to a second vessel. In an aspect, the purification step may be conducted in the second vessel. An organic solvent and a basic aqueous solution may be added to the second vessel. The basic solution may be added to remove excess concentrated acid. The organic solvent may be added to extract the organic compounds in the product mixture into the organic phase. The compounds may include tricaprilin, monoesters, diesters, tetraesters, 3-MCPD, 2-MCPD, glycidol, or any combination thereof.

The second vessel may be maintained at a second temperature. In some aspects, the second temperature may range from about 25° C. to about 50° C. For example, the first temperature may be about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 33° C., about 44° C., about 45° C., about 46° C., about 47° C., about 48° C., about 49° C., or about 50° C.

In some aspects, the second vessel may be maintained at an atmospheric pressure.

In some aspects, the organic solvent in step (c) may include an ether. Suitable ethers include but are not limited to dimethoxyethane, ditheyl ether, methyl tert-butyl ether, dioxane, THF, 2-methylTHF, CPME, 4-MeTHP, or combinations thereof.

In some aspects, any suitable basic salt may be used to obtain a basic solution for use in step (c). In some aspects, the aqueous solution of a basic salt in step (c) may include a solution of sodium carbonate.

In some aspects, the purification step may further include removing a second in process sample from the second vessel and measuring the conductivity of the aqueous layer. In process samples may be taken under the desired conductivity is reached. In some aspects, the desired conductivity may be less than 100 uS/cm.

After the desired conductivity is reached, the product mixture may be subject to charcoal treatment. In some aspects, charcoal may be added to the second vessel. The charcoal treatment may be conducted at room temperature at 3 g/min.

The next step in the purification process may include removing organic solvent from the product mixture. In some aspects, the organic solvent may be removed by distillation. The distillation may be conducted at a third temperature and pressure. In some aspects, the third temperature may range from about 40° C. to about 80° C. For example, the first temperature may be about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C. In some aspects, the pressure may be 1 mbar.

In some aspects, a second in process sample may be removed during the distillation step to measure the organic solvent remaining in the product mixture. The distillation may be continued until the amount of organic solvent in the product mixture is 500 ppm or less. The amount of solvent may be less than about 500 ppm, less than about 400 ppm, less than about 100 ppm, less than about 50 ppm or less than about 10 ppm.

In some aspects, the purification step may further include a recrystallization step. In an aspect, the recrystallization step may comprise the steps of: adding a polar solvent to the tricaprilin and impurities forming a second mixture comprising a polar solvent, tricaprilin, and impurities, cooling the second mixture to a third temperature, adding seeding crystals to the second mixture forming a third mixture comprising seeding crystals and the second mixture, stirring the third mixture for a third specified time at a third temperature, cooling the third mixture to a fourth temperature in a fourth specified time, maintaining the third mixture at the fourth temperature for a fifth specified time, adding water to the third mixture, maintaining the third mixture at the fourth temperature, filtering the third mixture on a filter cooled at the fourth temperature, washing the third mixture with an aqueous solution of the polar solvent at a fifth temperature, repeating the washing step, collecting crystals of tricaprilin, and evaporating residual solvent from the crystals using a rotary evaporator.

In an aspect, the recrystallization step may include adding one or more polar solvent to the product mixture and cooling the product mixture to a fourth temperature. In some aspects, the fourth temperature may range from about −20° C. to about 0° C. For example, the first temperature may be about −20° C., about −19° C., about −18° C., about −17° C., about −16° C., about −15° C., about −14° C., about −13° C., about −12° C., about −11° C., about −10° C., about −9° C., about −8° C., about −7° C., about −6° C., about −5° C., about −4° C., about −3° C., about −2° C., about −1° C., or about 0° C.

The one or more polar solvents may include water, acetone, acetonitrile, dimethylformamide (DMF), dimelthylsulfoxide (DMSO), isopropanol, methanol, or a combination thereof. In some aspects, the one or more polar solvents may be a mixture of water and methanol.

To obtain a composition of ultra-pure tricaprilin, the solvent may be removed from the product mixture by heating the product mixture to a fifth temperature. In some aspects, the fifth temperature may range from about 40° C. to about 80° C. For example, the fifth temperature may be about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C. In some aspects, the pressure may be 1 mbar.

### III. Pharmaceutical Compositions

Further aspects of the present disclosure are directed to pharmaceutical compositions comprising the ultra-pure tricaprilin provided herein. These pharmaceutical compositions may comprise one or more pharmaceutically appropriate carriers or excipients. The ultra-pure tricaprilin provided herein may be utilized as one or more active pharmaceutical ingredients in a pharmaceutical composition. These pharmaceutical compositions may be used in prophylactic or therapeutic methods of treating various diseases or disorders as described herein below. Alternatively, or in addition, these pharmaceutical compositions may be prepared as a food product and/or the nutritive composition which may, in an aspect, be a dietary supplement, a functional food, a novel food, a food additive, a food supplement, a dietary food, a power snack, an appetite suppressant or a strength and/or endurance sports supplement. In other instances, the ultra-pure tricaprilin provided herein may be used as an excipient in a pharmaceutical composition comprising one or more active pharmaceutical ingredients.

### Pharmaceutically Acceptable Carriers and Excipients

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. In various aspects, compositions disclosed herein may further compromise one or more pharmaceutically acceptable diluent(s), excipient(s), or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art. Pharmaceutical compositions may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, and combinations thereof. Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, Pa., latest edition, which is incorporated herein by reference.

In various aspects, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In other aspects, any of the well-known techniques, carriers, and excipients may be used as suitable and as understood in the art.

In various aspects, pharmaceutical compositions described herein may be an aqueous suspension comprising one or more polymers as suspending agents. In some aspects, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In other aspects, compositions disclosed herein may comprise at least 5%, at least 10%, at least 20%, at least 25%, at least 30%, at least 35%, at least 40%, at least 45%, at least 50% total amount of polymers as suspending agent(s) by total weight of the composition. [0189] In various aspects, pharmaceutical compositions disclosed herein may comprise a viscous formulation. In some aspects, viscosity of the composition may be increased by the addition of one or more gelling or thickening agents. In other aspects, compositions disclosed herein may comprise one or more gelling or thickening agents in an amount to provide a sufficiently viscous formulation to remain on treated tissue. In still other aspects, compositions disclosed herein may comprise at least 5%, at least 10%, at least 20%, at least 25%, at least 30%, at least 35%, at least 40%, at least 45%, at least 50% total amount of gelling or thickening agent(s) by total weight of the composition. In yet other aspects, suitable thickening agents can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethylcellulose (CMC), silicon dioxide, polyvinylpyrrolidone (PVP: povidone), Splenda® (dextrose, maltodextrin and sucralose), or combinations thereof. In some aspects, suitable thickening agent may be carboxymethylcellulose.

In various aspects, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least 5%, at least 10%, at least 20%, at least 25%, at least 30%, at least 35%, at least 40%, at least 45%, at least 50% total amount of one or more agents by total weight of the composition. In other aspects, one or more of these agents may be added to improve the performance, efficacy, safety, shelf-life and/or other property of the muscarinic antagonist composition of the present disclosure. In s aspects, additives will be biocompatible, and will not be harsh, abrasive, or allergenic.

In various aspects, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In other aspects, compositions disclosed herein may comprise at least 5%, at least 10%, at least 20%, at least 25%, at least 30%, at least 35%, at least 40%, at least 45%, at least 50% total amount of one or more acidifying agents by total weight of the composition.

In various aspects, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In other aspects, compositions disclosed herein may comprise at least 5%, at least 10%, at least 20%, at least 25%, at least 30%, at least 35%, at least 40%, at least 45%, at least 50% total amount of one or more alkalizing agents by total weight of the composition.

In various aspects, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art. In some aspects, compositions disclosed herein may comprise at least 5%, at least 10%, at least 20%, at least 25%, at least 30%, at least 35%, at least 40%, at least 45%, at least 50% total amount of one or more antioxidants by total weight of the composition.

In other aspects, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In another aspect, compositions disclosed herein may comprise at least 5%, at least 10%, at least 20%, at least 25%, at least 30%, at least 35%, at least 40%, at least 45%, at least 50% total amount of one or more buffering agents by total weight of the composition. In other aspects, the amount of one or more buffering agents may depend on the desired pH level of a composition. In some aspects, pharmaceutical compositions disclosed herein may have a pH of about 6 to about 9. In other aspects, pharmaceutical compositions disclosed herein may have a pH greater than about 8, greater than about 7.5, greater than about 7, greater than about 6.5, or greater than about 6. In a preferred aspect, compositions disclosed herein may have a pH greater than about 6.8.

In various aspects, pharmaceutical compositions disclosed herein may comprise one or more preservatives. As used herein, “preservatives” refers to agents or combination of agents that inhibits, reduces, or eliminates bacterial growth in a pharmaceutical dosage form. Non-limiting examples of preservatives include Nipagin, Nipasol, isopropyl alcohol and a combination thereof. In some aspects, any pharmaceutically acceptable preservative can be used. In other aspects, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition.

In other aspects, pharmaceutical compositions disclosed herein may comprise one or more surface-acting reagents or detergents. In some aspects, surface-acting reagents or detergents may be synthetic, natural, or semi-synthetic. In other aspects, compositions disclosed herein may comprise anionic detergents, cationic detergents, zwitterionic detergents, ampholytic detergents, amphoteric detergents, nonionic detergents having a steroid skeleton, or a combination thereof. In still other aspects, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition.

In various aspects, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some aspects, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition.

In other aspects, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In preferred aspects, a vapor pressure depression method is used to calculate the osmolarity of the compositions disclosed herein. In some aspects, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In other aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some aspects, a pharmaceutical composition described herein has an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In still other aspects, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition.

### Dosage Formulations

Suitable routes of administration may, for example, include oral, rectal, transmucosal, especially trans-nasal, intestinal or parenteral delivery, including intramuscular, subcutaneous and intramedullary injections as well as, intravenous, intraperitoneal, intranasal injections, intraocular (e.g., via eye drops) or topical (e.g., creams or ointments). Due to the ultra-purity of the tricaprilin disclosed herein, it is especially suitable for oral administration.

Pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

Pharmaceutical compositions suitable for use in context of the present disclosure include compositions wherein the active ingredients are contained in an amount effective to achieve the intended purpose. In some aspects, a therapeutically effective amount means an amount of active ingredients (i.e., the ultra-pure tricaprillin disclosed herein) effective to prevent, slow, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the present disclosure, the therapeutically effective amount or dose can be estimated initially from in vitro and in vivo assays and or screening platforms disclosed herein. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition.

Dosage amount and interval may be adjusted individually to brain or blood levels of the active ingredient that are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, etc. Effective doses may be extrapolated from dose-responsive curves derived from in vitro or in vivo test systems.

### IV. Methods of Treatment

As noted above, the present disclosure provides for an ultra-pure composition of tricaprilin. This ultra-pure composition is particularly appropriate for applications requiring high purity grades and at the same time high active ingredient levels, e.g., for nutritional and for pharmaceutical applications and uses (e.g., for applications in high dosages and/or with high concentrations).

Accordingly, in various aspects, a method of treating a subject in need thereof is provided. In an aspect, a method of prophylactically or therapeutically treating a disease in a subject is provided. In an aspect the disease to be treated (prophylactically or therapeutically) may be associated with a disorder of the energy metabolism, especially ketone body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus, migraine and migraine headaches, epilepsy, especially infantile spasms as well as effects or side-effects of chemotherapy. In some aspects, a method of prophylactically or therapeutically treating Alzheimer's disease, especially mild to moderate forms of Alzheimer's disease; migraine and migraine headaches; as well as epilepsy, especially infantile spasms in a subject in need thereof.

In accord with the foregoing, a pharmaceutical composition comprising the ultra-pure tricaprilin is provided for the use of prophylactically or therapeutically treating a subject with a disease is provided, where the disease is be associated with a disorder of the energy metabolism, especially ketone body metabolism, such as especially craniocerebral trauma, stroke, hypoxia, cardiovascular diseases such as myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytomas and glioblastomas, HIV, rheumatic diseases such as rheumatoid arthritis and arthritis urica, diseases of the gastrointestinal tract such as chronic inflammatory bowel diseases, especially ulcerative colitis and Crohn's disease, lyosomal storage diseases such as sphingolipidosis, especially Niemann-Pick disease, diabetes mellitus, migraine and migraine headaches, epilepsy, especially infantile spasms as well as effects or side-effects of chemotherapy. In some aspects, a pharmaceutical composition comprising the ultra-pure tricaprilin is provided for use in prophylactically or therapeutically treating Alzheimer's disease, especially mild to moderate forms of Alzheimer's disease; migraine and migraine headaches; as well as epilepsy, especially infantile spasms in a subject in need thereof.

In accord with the foregoing, in an aspect a high dose of ultra-pure tricaprilin is administered to a subject in need. The high dose may be administered according to any of the dose formulations described above. In particular aspects, the high does may be administered orally. In some aspects, the high dose may be administered systemically. In an aspect, a suitable dosage regimen may comprise administering at least about 20 mg/day to about 100 mg/day of the ultra-pure tricaprilin to a subject in need thereof. In an aspect, a suitable dosage regimen may comprise administering at least 20 mg, 21 mg, 22 mg, 23 mg, 24 mg, 25 mg, 26 mg, 27 mg, 27 mg, 28 mg, 29 mg, 30 mg, 31 mg, 32 mg, 33 mg, 34 mg, 35 mg, 36 mg, 37 mg, 38 mg, 39 mg, 40 mg tricaprilin per day. The tricaprilin composition may be combined with suitable excipients or combined with other active ingredients, such that the total amount of tricaprilin ingested per day is in the range of 20 mg/day to 100 mg/day. Further, several divided dosages as well as staggered dosages, can be administered daily or sequentially, or the dose can be continuously infused, or can be a bolus injection. Further, the dosages of the compound(s) described herein can be proportionally increased or decreased as indicated by the exigencies of the therapeutic or prophylactic situation. The selected dosage level will depend upon a variety of factors including the route of administration, the time of administration, and the rate of excretion, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

If desired, the effective daily dose of the active compound may be administered as two, three, four, five, six or more sub-doses administered separately at appropriate intervals throughout the day, optionally, in unit dosage forms. Similarly, the daily dose may be administered for any suitable duration, for example for at least a week, 2-weeks, 3-weeks, 1-month, 2-month, 3-month, 4 months, 5-months, 6-months, 7-months, 8-months, 9-months, 10-months, 11-months, 1 year or more.

While it is possible for a compound described herein to be administered alone, it may be administered in combination with other active composition.

By the term “combination” is meant either a fixed combination in one dosage unit form, or a kit of parts for the combined administration where a composition described herein and a combination partner may be administered independently at the same time or separately within time intervals that especially allow that the combination partners show a cooperative, e.g., synergistic, effect, or any combination thereof. The compositions described herein may be administered, simultaneously or sequentially, with an anti-inflammatory, antiproliferative, antibiotics, NSAIDs, painkillers, chemotherapeutic agent, immunosuppressant, other anti-cancer drugs, cytotoxic agent or salt thereof.

As a person skilled in the art will recognize from the previous detailed description and from the figures and claims, modifications and changes may be made to the aspects of the invention without departing from the scope of this invention as defined in the following claims.

### EXAMPLES

### Example 1: Experiments

Tricaprilin is synthesized by condensation of glycerol under acidic conditions with three equivalents of octanoic acid (caprylic acid), the latter being used in excess. Octanoic acid is treated with H₂SO₄-conc, and the mixture is heated to approx. 115° C. Glycerol is charged slowly and the mixture is then stirred overnight. Subsequently, the mixture is cooled to room temperature and diluted with MTBE (or an alternative solvent that does not lead to methyl esters in a subsequent batch). The solution is then filtered over active charcoal for decolorization. The organic phase is washed several times with Na₂CO₃-aq. to deplete residual octanoic acid and concentrated H₂SO₄, followed by extractions with water to remove any residual salts. Afterwards MTBE is distilled off under reduced pressure to obtain Tricaprilin as a clear colorless liquid (see FIG. 5).

Experiments were conducted to test the significance of various parameters and novel steps were included to improve yield and purity of the samples. Table 4 provides descriptions and analysis of the experiments conducted and the results obtained. The Examples below describe the conclusions from these sets of experiments and describe in detail the unexpected nature of some of the findings.

### Example 2: Minimizing Condensation of Caprylic Acid on the Condenser

In the initial experiments (EXP-23-AA6314, EXP-23-AA6315, EXP-23-AA6317, EXP-23-AA6318, EXP-23-AA6319), an undesired condensation of the caprylic acid on the condenser between the reactor and the water receiving flash was observed. This phenomenon was considered critical not only because it reduced the amount of reagents available for the reaction, but also because it could lead to temporary blockage of the vacuum line (as in EXP-23-AA6317), reducing the efficacy of the water removal and leading to a potentially dangerous overpressure of the reactor caused by the nitrogen inlet. This observation was unexpected since the boiling point of the reagent at different pressure values (reported in Table 2) was always above the temperature of the reaction.

In the attempt to solve this issue, the addition of premixed Glycerol (glycerol) and octanoic acid were tested, (EXP-23-AA6320). This reduced the total residence time of the caprylic acid in the reactor. This approach did not avoid the condensation of the reagent on the condenser; additionally, it lead to a biphasic mixture during addition and during the reaction, due to the low miscibility of these reagents.

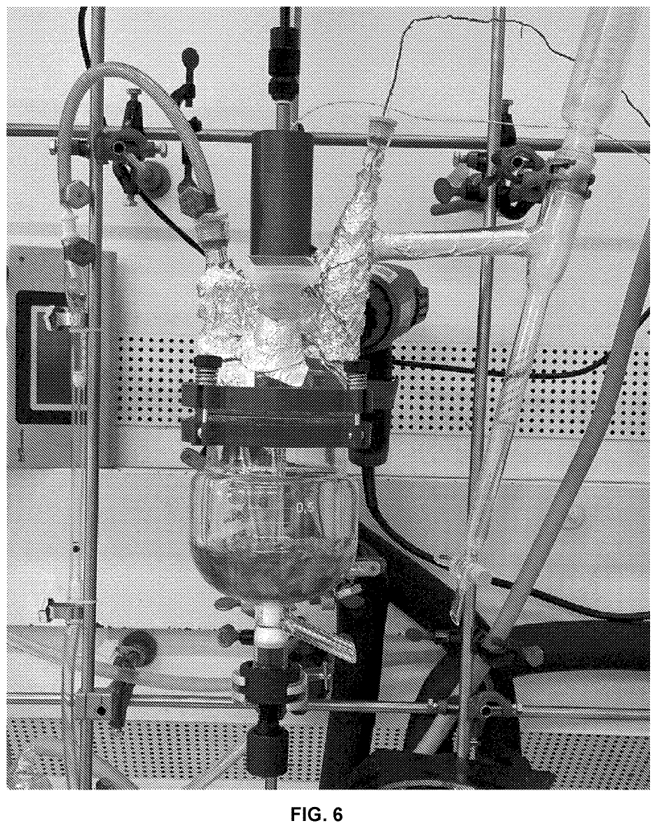

Decreasing the nitrogen flow from 60 to 6 NL/h was not sufficient to resolve this problem (EXP-23-AA6320). Surprisingly using an apparatus with a longer tube, for example a Dean Stark apparatus instead of a Dimroth cooler (EXP-23-AA-8203), suggesting the importance of the apparatus geometry. Using a Dean Stark apparatus resulted in a longer way for the vapors to go before reaching the condenser; consequently, the caprylic acid recondensed in the reaction mixture before leaving the reactor to reach the condenser. Water, on the other hand, at the given values of temperature and pressure, is sufficiently volatile to be removed from the reaction mixture. In almost all the experiments where the Dead Stark apparatus was used, in addition to the water released from the reaction, the amounts of caprylic acid recollected were in the order of few mL, ensuring the excess of caprylic acid in the reactor. A representation of the apparatus used in the reported in FIG. 6.

### Example 3: Maximizing Conversion to Product

### Mode of Addition of Glycerol (Glycerol): From Above

If chemicals do not leave the reactor, the reaction can proceed to completion upon gradual formation of mono ester, di ester and ultimately of the product, glyceryl trioctanoate. Glycerol, the limiting reagent, was dosed from above into a stirred and heated mixture of octanoic acid and sulfuric acid. The dosing speed under these conditions strongly influenced the tetraester formation; this impurity was quantified beyond the originally specified limit of 0.4% in almost all the experiments. Decreasing the catalyst loading to 0.5% is not sufficient to reach specification limit (see Table 6).

TABLE 6
Overview of results when pure glycerol is added from above the heated and stirred
octanoic acid/sulfuric acid mixture.

| | | | Dosing | H₂SO₄ | TRICAPRILIN | |
|---|---|---|---|---|---|---|
| | Reaction | Temperature | speed | loading | Purity | Tetraesters |
| Entry | name | [° C.] | [mL/h] | [% mol] | [% a] | [% a] |
| 1 | EXP-23- | 130 | 2.6 | 1 | 87.01 | 1.05 |
| | AA6315 | | | | | |
| 2 | EXP-23- | 130 | 2.1 | 1 | 97.81 | 0.74 |
| | AA6317 | | | | | |
| 3 | EXP-23- | 115 | 8 | 1 | 75.36 | 6.53 |
| | AA6314 | | | | | |
| 4 | EXP-23- | 115 | 3 | 1 | 83.85 | 2.77 |
| | AA6318 | | | | | |
| 5 | EXP-23- | 115 | 1 | 1 | 95.92 | 1.05 |
| | AA6319 | | | | | |
| 6 | EXP-23- | 105 | 2 | 1 | 94.6 | 0.69 |
| | AA6322 | | | | | |
| 7 | EXP-23- | 105 | 3 | 0.5 | 96.19 | 1.85 |
| | AA6324 | | | | | |
| 8 | EXP-23- | 105 | 3 | 1 | 95.3 | 0.11 |
| | AA8211 | | | | | |
| 9 | EXP-23- | 95 | 1 | 1 | 95.45 | 0.07 |
| | AA8212 | | | | | |

The experiments where the tetra ester level was below the original specification of 0.4% are as listed in the last entries 8 and 9. In general, the head of the reactor in the lab scale requires additional heating due to the distance from the heated jacket. When this additional heating is absent, the water produced from the reaction is removed more slowly and the tetra ester formation is inhibited (EXP-23-AA8211). Additionally, if the reaction temperature is reduced to 95° C. and the addition speed of pure glycerol is reduced to 1 mL/h, the reaction proceeds with neglectable by-product formation product (EXP-23-AA8212).

### Mode of Addition of Glycerol (Glycerol): From Above

Addition of glycerol below the reaction surface unexpectedly resulted in an improved mixing with reaction components, resulting in a decreased tetraester formation. All the experiments carried out using such addition method resulted in tetraester levels below 0.5%; Addition of glycerol directly in the reaction mixture resulted in an overall increase of the robustness of the reaction. Fast addition did not result in an strong by-product increase (EXP-23-AA8208); increasing the catalyst loading to 4.5% mol resulted in a only moderate tetraester increase (EXP-23-AA8206) where lower catalyst loading had basically no apparent influence (EXP-23-AA6326). Temperature appears to be less critical as well: comparable results in terms of yield and purity have been obtained when the reaction was carried out at 115, 105 or 95° C. Surprisingly, decreasing the reaction temperature from 115° C. to 105° C. with fast addition (EXP-23-AA8210) had a small but positive effect on the by-product formation and no influence on the conversion. Decreasing at the same time temperature and catalyst yield did not result in noticeable change in the purity profile and final yield of the product (EXP-23-AA6327); unexpectedly, in this case the tetraester detected in the final product was relatively high if compared with the other experiments in this series (Table 7).

TABLE 7
Overview of results when pure glycerol is added directly within the heated and stirred
octanoic acid/sulfuric acid mixture.

| | | | Dosing | H₂SO₄ | TRICAPRILIN | |
|---|---|---|---|---|---|---|
| | Reaction | Temperature | speed | loading | Purity | Tetraesters |
| Entry | name | [° C.] | [mL/h] | [% mol] | [% a] | [% a] |
| 1 | EXP-23- | 115 | 8 | 1 | 98.4 | 0.17 |
| | AA8208 | | | | | |
| 2 | EXP-23- | 115 | 1 | 4.5 | 97.55 | 0.44 |
| | AA8206 | | | | | |
| 3 | EXP-23- | 115 | 3 | 1 | 98.02 | 0.20 |
| | AA6325 | | | | | |
| 4 | EXP-23- | 115 | 3 | 0.5 | 98.47 | 0.22 |
| | AA6326 | | | | | |
| 5 | EXP-23- | 105 | 8 | 1 | 98.24 | 0.15 |
| | AA8210 | | | | | |
| 6 | EXP-23- | 95 | 3 | 0.5 | 97.73 | 0.44 |
| | AA6327 | | | | | |

### Addition Mode of Glycerol: Above but Diluted

In order to allow the dosing from above but to keep the tetraesters level low, it was rationalized that decreasing the starting concentration of Glycerol would reduce the possibility of acid catalyzed ether formation. Hence, Glycerol was diluted with an equivalent amount of water and then added dosed to the reaction; the additional water would be removed just as the water formed during the reaction to TRICAPRILIN. This approach resulted in a more controlled tetraester formation in all the experiments, as summarized in the table below.

Results suggest that tetraester formation can be minimized if the dosing speed is low (EXP-23-AA6328). Increasing the dosing speed (EXP-23-AA3527 or EXP-23-AA6332) or decreasing the catalyst loading (EXP-23-AA358214) do not result in an improve in the quality of the material. A non-exhaustive list of experiment with this dosing mode is summarized in Table 8.

TABLE 8
Non exhaustive overview of results after dosing the glycerol/water mixture

| | | | Dosing | H₂SO₄ | TRICAPRILIN | |
|---|---|---|---|---|---|---|
| | | Temperature | speed | loading | Purity | Tetraesters |
| Entry | Experiment | [° C.] | [mL/h] | [% mol] | [% a] | [% a] |
| 1 | EXP-23- | 100 | 3 | 1 | 98.59 | 0.11 |
| | AA6328 | | | | | |
| 2 | EXP-23- | 100 | 3.5 | 1 | 97.64 | 0.21 |
| | AA3527 | | | | | |
| 3 | EXP-23- | 100 | 5 | 1 | 94.64 | 0.24 |
| | AA6332 | | | | | |
| 4 | EXP-23- | 100 | 3 | 0.5 | 98.29 | 0.16 |
| | AA8214 | | | | | |

### Use of Other Acid Catalysts

Phosphoric acid (85%) was tested as an alternative to sulfuric acid used in other experiments. The conversion to the product was much slower and after almost 23 h of reaction at 100° C. and subsequent workup, analysis of isolated material showed 75% a of diesters and only 20% product (EXP-23-AA6330).

TABLE 9
Reaction outcome when using phosphoric acid instead of sulfuric acid.

| | | Dosing | H₃PO₄ | Sum of | TRICAPRILIN | |
|---|---|---|---|---|---|---|
| | Temp. | speed | loading | Diester | Purity | Tetraesters |
| Experiment | [° C.] | [mL/h] | [% mol] | [% a] | [% a] | [% a] |
| EXP-23- | 100 | 6 | 1 | 75 | 20 | n.d. |
| AA6330 | | | | | | |

Glycidol amount is influenced by the combination of different parameters such as the quality of the starting materials as well as the applied reaction conditions. In general, its formation requires harsh conditions, in order to form the strained 3-member ring of the epoxide. As observed in a few experiments, a higher reaction temperature was associated with an increase in the glycidol levels (EXP-23-AA6319 and EXP-23-AA8206).

Additional considerations can be made: although milder conditions (EXP-23-AA8212) and slower glycerol addition (EXP-23-AA8209) mitigate its formation, glycidol is formed if the catalyst loading is increased, even if slow addition (EXP-23-AA8206) or increase temperature (EXP-23-AA6319 and EXP-23-AA8206) is applied. Decreasing the catalyst loading appears to have a beneficial effect (EXP-23-AA6322 and AA6324) even when the dosing speed was slightly increased.

Quite interestingly, mixing glycerol with water appears to be useful in reducing the glycidol formation and an increased dosing speed does not correlate to an increase in this side product (EXP-23-AA6328 and EXP-23-AA8214).

It might be worth to note that despite certain trend can be identified, these values are not always consistent due to the very low amount considered and the high sensitivity of the measure. These results are summarized in the following Table 10.

TABLE 10
Effect of reaction condition on glycidol formation

| | | Addition of | | Catalyst | | |
|---|---|---|---|---|---|---|
| | Addition of | Glycerol | Temperature | loading | Reaction | Glycidol |
| Experiment | Glycerol | [mL/h] | [° C.] | [% mol] | Time | [ppb] |
| EXP-23- | above | 1 | 115 | 1 | 17 h | 47700 |
| AA6319 | | | | | | |
| EXP-23- | above | 1 | 95 | 1 | 20 h | 1048 |
| AA8212 | | | | | | |
| EXP-23- | below | 3 | 95 | 0.5 | 19 h | 3506 |
| AA6327 | | | | | | |
| EXP-23- | above | 2 | 105 | 1 | 23 h | 7677 |
| AA6322 | | | | | | |
| EXP-23- | above | 3 | 105 | 0.5 | 23 h | 4500 |
| AA6324 | | | | | | |
| EXP-23- | below | 1.5 | 105 | 1 | 15 h | 1044 |
| AA8209 | | | | | | |
| EXP-23- | below | 1 | 115 | 4.5 | 20 h | 31400 |
| AA8206 | | | | | | |
| EXP-23- | above, with | 3 | 100 | 1 | 19 h | 1610 |
| AA6328 | water | | | | | |
| EXP-23- | above, with | 3 | 100 | 0.5 | 19 h | 1858 |
| AA8214 | water | | | | | |

### Example 4: Work-Up Procedure

During the workup, treatment of the reaction mixture with a basic aqueous solution allow to quench the acidity and wash the excess of octanoic acid by converting it to the more water-soluble sodium octanoate. Experiments were conducted to test various work-up procedures.

### Different Base

In addition to the intensively used sodium carbonate, sodium bicarbonate was evaluated as well. NaOH was not tested due to the increased risk of saponification represented by treating an ester with a such reagent. Experimental results showed that natrium bicarbonate does not remove promptly the fatty acid excess, resulting in a very high acid value in the final product. Table 11 provides a comparison between these two reagents.

TABLE 11
Removal of octanoic acid during the work up by sodium
carbonate and bicarbonate (constant conditions).

| | Organic | | Number of | Acid value |
|---|---|---|---|---|
| Experiment | phase | Base | washings | [mg KOH/g] |
| EXP-23-AA6328 | MtBE | Na₂CO₃ | 4 | 0.16 |
| EXP-23-AA8211 | MtBE | NaHCO₃ | 4 | 17.79 |

The effect of different basic washing solutions such as, K2CO3, KHCO3 and NaHCO₃) at 40° C. in the glycidol and MCPD removal was evaluated as well. Results are summarized in Table 12.

TABLE 12
Effect of different bases on the MCPD and glycidol removal.

| | | 3-MCPD | 2- MCPD | Glycidol |
|---|---|---|---|---|
| Experiment | Base | [ppb] | [ppb] | [ppb] |
| EXP-23-AA2362 | Starting Values | 184 | 66 | 1040 |
| EXP-23-AA4115-1 | 10% K₂CO₃ | 204 | 75 | 522 |
| EXP-23-AA4115-2 | 10% KHCO₃ | 196 | 73 | 574 |
| EXP-23-AA4115-3 | 10% NaHCO₃ | 197 | 71 | 558 |

Different bases tested have no impact on the removal of MCPD and little impact on the glycidol amount. Considering the reduced efficacy of sodium bicarbonate in the removal of excess of octanoic acid during the work up, natrium carbonate was identified as base of choice.

### Different Solvents

Next, the organic solvent during the workup was investigated. The reaction outcome was split in two and the same procedure was applied for both the fractions. Toluene and heptane performances were compared and results are summarized in Table 13.

TABLE 13
Purity profile of TRICAPRILIN after workup with heptane or toluene.

| | | | Sum of | | | | | |
|---|---|---|---|---|---|---|---|---|
| | Organic | Monoester | Diester | TRICAPRILIN | Tetraester | | | |
| Experiment | phase | [% a] | [% a] | [% a] | [% a] | MCPD | MCPD | Glycidol |
| EXP-23- | Heptane | n.d. | 0.09 | 97.81 | 0.76 | 4500 | 430 | 69600 |
| AA6317 | (100 g) | | | | | | | |
| | Toluene | n.d. | 0.15 | 97.73 | 0.76 | 4840 | 601 | 67800 |
| | (100 g) | | | | | | | |

No change in the purity profile of the sample was observed; it is therefore concluded that both of them are equally suitable.

### No Solvent

A workup was attempted without any additional organic solvent as well (EXP-23-AA6318). In this case, different washings solutions were tested: (a) very good phase separation within seconds was obtained using a mixture of saturated NaCl and 10% Na₂CO₃. The high amount of salt contained ensured fast phase separation and clear phases; (b) The basic wash was followed by treating the reaction crude with a NaCl saturated aqueous solution. phase separation was very good and achieved within seconds; and (c) the organic residue was washed with only water. In this case, after 30 minutes the organic layer was still turbid and contained copious amount of water.

It was concluded that during the workup, if no organic solvent is present, water phase with a strong ionic strength (e.g., NaCl solution) is needed to achieve a satisfactory phase separation.

### Example 5: Refining

After workup, the material can be further processed to improve its appearance, odor or purity profile. Different materials and techniques were tested and evaluated.

### Activated Charcoal

The activated charcoal treatment allows to remove impurities in the material, improving its appearance. Different types of active charcoal have been tested under different conditions. The results as summarized in Table 14.

TABLE 14
Summary of the different active charcoal filter used.

| | | | | | | 3- | 2- | |
|---|---|---|---|---|---|---|---|---|
| | | | | Amount | Flow | MCPD | MCPD | Glycidol |
| Experiment | Material | Conditions | Temp. | processed | (ml/min) | [ppb] | [ppb] | [ppb] |
| EXP-23- | Starting | | | | | 231 | <100 | 465 |
| AA2366 | Material | | | | | | (78) | |
| EXP-23- | Carbofil CA | concent | RT | 15 mL | 3 | 192 | <100 | <100 |
| AA5430 | | rated | | | | | | |
| | | 50% in | RT | 15 mL | 6 | 196 | <100 | 215 |
| | | MtBE | | | | | | |
| | Carbofil PA | concent | RT | 15 mL | 3 | 202 | <100 | 246 |
| | | rated | | | | | | |
| | | 50% in | RT | 15 mL | 6 | 204 | <100 | 224 |
| | | MtBE | | | | | | |
| | Carbofil PXs | concent | RT | 15 mL | 3 | 195 | <100 | 285 |
| | | rated | | | | | | |
| | | 50% in | RT | 15 mL | 6 | 197 | <100 | 234 |
| | | MtBE | | | | | | |
| EXP-23- | Starting | | | | | 197 | <100 | 1092 |
| AA2370 | Material | | | | | | (56) | |
| EXP-23- | Carbofil CA | concent | 70° C. | 150 g | ~4 | 177 | <100 | 354 |
| AA5433 | | rated | | | g/min | | (58) | |
| | Carbofil PA | concent | 70° C. | 150 g | ~3 | 168 | <100 | 295 |
| | | rated | | | g/min | | (62) | |
| | Carbofil PXS | concent | 70° C. | 150 g | ~1.5 | 208 | <100 | 278 |
| | | rated | | | g/min | | (60) | |
| EXP-23- | Starting | | | | | 122 | <100 | 15109 |
| AA3341 | Material | | | | | | (45) | |
| EXP-23- | BECODISC | concent | RT | 7 mL | 1 | 1615 | 678 | 2914 |
| AA3343 | | rated | | | drop/sec | | | |
| | BECODISC | 50% in | RT | 14 mL | 1 | 1515 | 598 | 9219 |
| | | MtBE | | | drop/sec | | | |

The best results were obtained treating a concentrated sample of TRICAPRILIN with CARBOFIL CA at RT (EXP-23-AA5430). The same material did not perform comparably when the temperature was increased to 70° C., but this might be caused by the amount treated, which was increased 10 times (EXP-23-AA5433) and might have led to a saturation of the pad. Treating TRICAPRILIN with BECODISC resulted in a decrease of glycidol levels and surprisingly to a strong increase in the MCPD amount. TRICAPRILIN was also treated with celite and active charcoal in powder at 110° C. overnight. The results are presented in Table 15.

TABLE 15
Treatment of TRICAPRILIN with celite and charcoal at 110° C.

| | | Sum of | | | 3- | 2- | |
|---|---|---|---|---|---|---|---|
| | | Diester | TRICAPRILIN | Tetraester | MCPD | MCPD | Glycidol |
| Experiment | Material | [% a] | [% a] | [% a] | [ppb] | [ppb] | [ppb] |
| EXP-23-AA2362 | Starting | 0.36 + 0.79 | 97.72 | 0.04 + 0.10 | 184 | 66 | 1040 |
| | Values | | | | | | |
| EXP-23 AA4115-4 | Celite (3 g) | 0.33 + 0.69 | 97.89 | 0.04 + 0.11 | 169 | 64 | <100 |
| (50 g TRICAPRILIN) | and Charcoal | | | | | | (89) |
| | (2.5 g) | | | | | | |

Interestingly, this treatment resulted in a marked decrease in the glycidol content. No impact on the quality of the material nor on the amount of chlorinated by-products was observed. In conclusion, treatment of concentrated TRICAPRILIN at RT with active charcoal can significantly decrease the amount of glycidol in the sample (EXP-23-AA4115-4 and EXP-23-AA5430). Unfortunately, none of the experiments influenced the MCPD species.

### Diatomaceous Earth

Analogous to active charcoal, diatomaceous earth are also commonly employed as filtering aid or adsorption material due to their high porosity and low density. Different types of material and conditions have been tested and the results are summarized in Table 16.

TABLE 16
Screening of different diatomaceous earth under different
conditions and the related impact on MCPD/Glycidol levels

| | | | 3- | 2- | |
|---|---|---|---|---|---|
| | | | MCPD | MCPD | Glycidol |
| Experiment | Material | Conditions | [ppb] | [ppb] | [ppb] |
| EXP-23-AA8209 | Start material | | 260 | 68 | 1044 |
| EXP-23-AA6323 | Bentonite (2 weight equivalents) | @ RT overnight | 266 | <100 (62) | 574 |
| EXP-23-AA6331 | Bentonite (12.5% w) + Active | @ 120° C./30 mbar | 326 | <100 (87) | 306 |
| | charcoal (7% w) | | | | |
| EXP-23-AA6323 | Zeolite (2 weight equivalents) | @ RT overnight | 247 | <100 (65) | 2601 |
| EXP-23-AA6331 | Zeolite(6% w) + Active charcoal (7% w) | @ 120° C./30 mbar | 340 | <100 (87) | 640 |
| EXP-23-AA6323 | Fullers Earth (2 weight equivalents) | @ RT overnight | 254 | <100 (68) | 1299 |
| EXP-23-AA6331 | Fullers Earth (6% w) + Active | @ 120° C./30 mbar | 346 | <100 (89) | 187 |
| | charcoal (7% w) | | | | |
| EXP-23-AA6323 | Mol. Sieves 12X (2 weight equivalents) | @ RT overnight | 248 | <100 (61) | 1035 |
| EXP-23-AA6331 | Mol. Sieves 12X (6% w) + Active | @ 120° C./30 mbar | 283 | <100 (68) | 317 |
| | charcoal (7% w) | | | | |
| EXP-23-AA3529 | Starting material | | 197 | <100 (56) | 1092 |
| | Tonsil210FF (10% w) | @ RT overnight | 176 | <100 (77) | 113 |
| | Tonsil310FF (10% w) | @ RT overnight | 184 | <100 (79) | 251 |
| | Vulcobent Protex Ü (10% w) | @ RT overnight | 172 | <100 (75) | 355 |
| | Vulcobent Aktiv G (10% w) | @ RT overnight | 171 | <100 (77) | 351 |
| | Sorbit R 40 (10% w) | @ RT overnight | 167 | <100 (75) | 351 |
| | Prosolv 730 (10% w) | @ RT overnight | 169 | <100 (67) | 317 |
| | Prosolv 50 (10% w) | @ RT overnight | 179 | <100 (74) | 377 |

The general observed trend is that the glycidol removal efficacy of all these materials increase with the temperature. The MCPD amount remains basically unvaried. In presence of activated charcoal and at increased temperature, the material that allowed glycidol removal at a bigger extend is the so called “Fullers Earth” (EXP-23-AA6331).

Another series of commercially available diatomaceous earth have been tested (EXP-23-AA3529). In general, this treatment resulted in a good decrease of the glycidol amount, with the best results given by the Tonsil210FF. MCPD species remained unchanged after all the treatments.

### Steam Distillation

Steam distillation was tested for the removal of volatile components, such as odors, by treating the material with a flow of water steam under vacuum conditions. Treating a sample of TRICAPRILIN of 51 g with 500 mL water at 130° C. with a flow of 10 mL/min resulted a strong decrease in the glycidol amount, as summarized in Table 17.

TABLE 17
Purity and MCPD/Glycidol content on a sample of TRICAPRILIN after steam distillation

| | Sum of | | Sum of | 3- | 2- | |
|---|---|---|---|---|---|---|
| | Diester | TRICAPRILIN | Tetraester | MCPD | MCPD | Glycidol |
| Experiment | [% a] | [% a] | [% a] | [ppb] | [ppb] | [ppb] |
| Starting | 0.36 + 0.79 | 97.72 | 0.04 + 0.10 | 184 | 66 | 1040 |
| Values (EXP- | | | | | | |
| 23-AA2362) | | | | | | |
| EXP-23- | 0.42 + 0.87 | 97.59 | 0.04 + 0.10 | 195 | 78 | 142 |
| AA6329 | | | | | | |

Steam distillation appear to be a suitable way to efficiently remove glycidol in TRICAPRILIN samples; MCPD levels remains unchanged. The purity profile remains comparable after the treatment.

### Ion Exchange

In an attempt to reduce the amount of MCPD and glycidol in the isolated TRICAPRILIN, the material was treated with different ion exchange resins at different conditions. The results are summarized in Table 18.

TABLE 18
TRICAPRILIN treatment with ion exchange resin at RT for prolonged time.

| | | | 3- | 2- | |
|---|---|---|---|---|---|
| | | | MCPD | MCPD | Glycidol |
| Experiment | Ion exchange | Conditions | [ppb] | [ppb] | [ppb] |
| EXP-23-AA8212 | Starting Values | | 172 | 40 | 1048 |
| EXP-23-AA3252-1 | Amberlite IRA 900 | RT@56 h | 203 | <100 (54) | 206 |
| EXP-23-AA3252-2 | Amberlite IRA 402 | RT@56 h | 185 | <100 (50) | 225 |
| EXP-23-AA3252-3 | Amberlite IRC 120 H | RT@56 h | 217 | <100 (58) | 1347 |

The use of anion exchange resin caused a decrease in glycidol but was accompanied by a slight increase in the MCPD species. The cation exchange resin used in EXP-23-AA3252-3 did not appear to be a suitable candidate for the removal of these impurities. Despite the positive results obtained with the anion exchange resins, their removal capability was insufficient to achieve levels of glycidol in the single-digit ppb.

The same resins were tested at an increase temperature (70° C.) but reduced reaction time (2 h) and results are summarized in Table 19.

TABLE 19
Treatment of TRICAPRILIN with ion exchange
resin at increased temperature for 2 hours.

| | | | 3-MCPD | 2-MCPD | Glycidol |
|---|---|---|---|---|---|
| Experiment | Ion exchange | Conditions | [ppb] | [ppb] | [ppb] |
| EXP-23-AA8208 | Starting Values | | 193 | <100 (85) | 11123 |
| EXP-23-AA3256-1 | Amberlite IRA 900 | 70° C. @ 2 h | 201 | <100 (92) | 3398 |

Despite the decrease in glycidol amount observed in the three experiments, the amount of 3-MCPC and 2-MCPD remains more or less constant. Overall, the treatment of TRICAPRILIN using ion exchange resins at increased temperature does not appear to be a valuable option.

### Short Path Distillation

TRICAPRILIN was subjected to a short path distillation with a jacket temperature of 130° C. and 4 mbar vacuum. The MCPD and glycidol profile of the sample was then analyzed, as summarized in Table 20.

TABLE 20
The MCPD and glycidol profile

| | 3-MCPD | 2- MCPD | Glycidol |
|---|---|---|---|
| Experiment | [ppb] | [ppb] | [ppb] |
| Starting material | 184 | <100 (66) | 1040 |
| EXP-23-AA3255 | 167 | <100 (59) | 4217 |

Considering that the glycidol level increased after the treatment, the use of short path distillation may not be advised as a purification method.

### Recrystallization

TRICAPRILIN can be recrystallized at a temperature close to 0° C. from MeOH. The presence of water as antisolvent is beneficial to the process in terms of yield and purity.

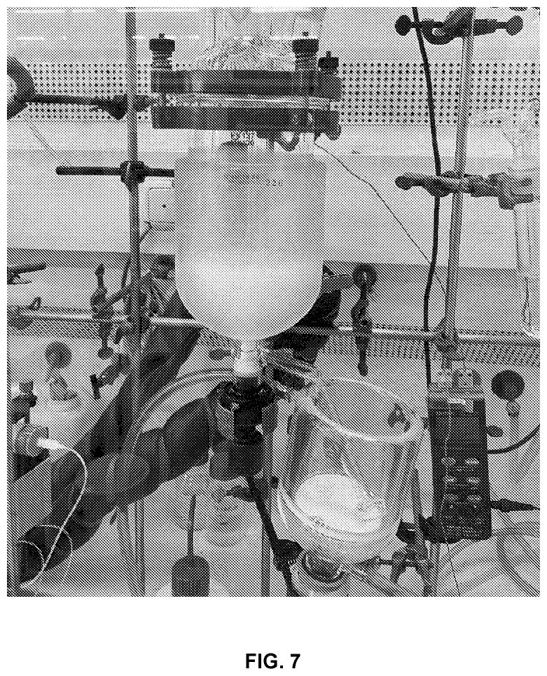

In general, the product is mixed with MeOH (8 Vol) and cooled to 0° C. Addition to seeding crystals was followed by controlled cooling from 0° to −10° C. within 3 hours. Water (1 Vol) is added slowly to the cold mixture and stirred for an additional hour. The crystals are then filtrated on a cooled filter and washed with a cold mixture of MeOH/Water (9:1). The crystals are collected and the solvent is removed at rotary evaporator. The set-up used is presented in FIG. 7 and the results are presented in Table 21.

TABLE 21
Exhaustive list of the crystallization experiment with purity and MCPD/glycidol content.

| | Yield of | | | | 3- | 2- | |
|---|---|---|---|---|---|---|---|
| | crystallization | Diester | TRICAPRILIN | Tetraester | MCPD | MCPD | Glycidol |
| Experiment | [%] | [% a] | [% a] | [% a] | [ppb] | [ppb] | [ppb] |
| EXP-23- | 51 | 0.02 | 99.54 | n.d. | 26 | 3 | 68 |
| AA8213-1 | | | | | | | |
| (small scale) | | | | | | | |
| EXP-23- | 31 | 0.06 | 99.44 | 0.02 | 120 | 29 | 183 |
| AA8213-2 | | | | | | | |
| (big scale) | | | | | | | |
| EXP-23- | 65 | n.d. | 98.6 | n.d. | n.d. | n.d. | <100 |
| AA8215 | | | | | | | (73) |
| (big scale, | | | | | | | |
| better cooling) | | | | | | | |
| EXP-23- | 71 | n.d. | 99.75 | 0.03 | 10 | n.d. | 1847 |
| AA8216 | | | | | | | |
| (From | | | | | | | |
| material | | | | | | | |
| with 31400 | | | | | | | |
| ppb | | | | | | | |
| glycidol) | | | | | | | |
| EXP-23- | 92 | n.d | 99.21 | 0.05 | 19 | n.d. | 188 |
| AA8217-1 | | | | | | | |
| (Stepwise | | | | | | | |
| cooling, | | | | | | | |
| 25% water | | | | | | | |
| added) | | | | | | | |
| EXP-23- | 96 | n.d. | 99.36 | 0.02 | 8 | n.d. | 90 |
| AA8217-2 | | | | | | | |
| (Second | | | | | | | |
| crystallization | | | | | | | |
| with same | | | | | | | |
| conditions) | | | | | | | |
| EXP-23- | 92 | n.d. | 99.43 | n.d. | n.d | n.d. | <100 |
| AA8218-1 | | | | | | | (70) |
| (Standard: | | | | | | | |
| 10% water | | | | | | | |
| at −10° C.) | | | | | | | |
| EXP-23- | 93 | n.d. | 99.57 | n.d. | <100 | <100 | <100 |
| AA8218-2 | | | | | (14) | (14) | (3) |
| (More pure | | | | | | | |
| starting | | | | | | | |
| material) | | | | | | | |
| EXP-23- | 90 | 0.04 | 99.17 | 0.05 | <100 | <100 | 146 |
| AA8218-3 | | | | | (50) | (23) | |
| (Water | | | | | | | |
| added at the | | | | | | | |
| beginning) | | | | | | | |
| EXP-23- | 88 | n.d. | 99.44 | n.d. | <100 | <100 | <100 |
| AA8218-4 | | | | | (20) | (17) | (13) |
| (High quality | | | | | | | |
| seeds) | | | | | | | |
| EXP-23- | 87 | n.d. | 99.40 | n.d. | <100 | <100 | <100 |
| AA8218-5 | | | | | (20) | (21) | (27) |
| (Water | | | | | | | |
| added at 0° C., | | | | | | | |
| then further | | | | | | | |
| cooled) | | | | | | | |
| EXP-23- | 88 | 0.23 | 96.2 | 0.11 | 117 | <100 | 335 |
| AA8218-6 | | | | | | | |
| (Using | | | | | | | |
| Celite to | | | | | | | |
| induce | | | | | | | |
| nucleation) | | | | | | | |
| EXP-23- | 92 | n.d. | 99.07 | n.d. | n.d. | n.d. | <100 |
| AA8219-1 | | | | | | | (55) |
| (Scale up) | | | | | | | |
| EXP-23- | 80 | n.d. | 99.26 | n.d. | n.d. | n.d. | <100 |
| AA8219-2 | | | | | | | (49) |
| (Using ACN | | | | | | | |
| instead of | | | | | | | |
| MeOH and | | | | | | | |
| 25% water) | | | | | | | |
| EXP-23- | 92 | n.d. | 99.37 | 0.01 | <100 | n.d. | <100 |
| AA8219-3 | | | | | (10) | | (30) |
| (Increasing | | | | | | | |
| water to 15%) | | | | | | | |
| EXP-23- | 87 | n.d. | 99.47 | n.d. | n.d. | n.d. | <100 |
| AA8219-4 | | | | | | | (61) |
| (Using | | | | | | | |
| EtOH | | | | | | | |
| instead of | | | | | | | |
| MeOH and | | | | | | | |
| 25% water) | | | | | | | |
| EXP-23- | 94 | n.d. | 99.65 | n.d. | n.d. | n.d. | n.d. |
| AA8221 | | | | | | | |
| (From | | | | | | | |
| MeOH at 0° C., | | | | | | | |
| then | | | | | | | |
| water | | | | | | | |
| addition at- | | | | | | | |
| 10° C.- | | | | | | | |
| standard | | | | | | | |
| conditions) | | | | | | | |
| EXP-23- | 94 | n.d. | 99.65 | n.d. | n.d. | n.d. | n.d. |
| AA8222 | | | | | | | |
| (From | | | | | | | |
| MeOH at 0° C., | | | | | | | |
| then water | | | | | | | |
| addition at- | | | | | | | |
| 10° C.) | | | | | | | |
| EXP-23- | n.a. | 0.03 | 99.57 | n.d. | n.d. | n.d. | 216 |
| AA8223-1 | | | | | | | |
| (Standard | | | | | | | |
| conditions, | | | | | | | |
| after 1 wash) | | | | | | | |
| EXP-23- | 91.1 | n.d | n.d. | n.d. | n.d. | n.d. | 126 |
| AA8223-2 | | | | | | | |
| (Standard | | | | | | | |
| condition, | | | | | | | |
| after 2 | | | | | | | |
| washings) | | | | | | | |
| EXP-23- | n.a | 0.28 | 99.36 | n.d. | n.d. | n.d. | 194 |
| AA8224-1 | | | | | | | |
| (Standard | | | | | | | |
| conditions, | | | | | | | |
| after 1 wash) | | | | | | | |
| EXP-23- | n.a. | 0.07 | 99.59 | n.d. | n.d. | n.d. | 124 |
| AA8224-2 | | | | | | | |
| (Standard | | | | | | | |
| conditions, | | | | | | | |
| after 2 | | | | | | | |
| wash) | | | | | | | |
| EXP-23- | 88 | n.d. | 99.66 | n.d. | n.d. | n.d. | <100 |
| AA8224-3 | | | | | | | (57) |
| (Standard | | | | | | | |
| conditions, | | | | | | | |
| after 3 wash) | | | | | | | |
| EXP-23- | 79 | 0.04 | 99.54 | 0.02 | n.d. | n.d | 130 |
| AA8225 | | | | | | | |
| (Standard | | | | | | | |
| conditions) | | | | | | | |
| EXP-23- | 76 | 0.02 | 99.63 | n.d. | n.d. | n.d. | 194 |
| AA8226 | | | | | | | |
| (Standard | | | | | | | |
| conditions) | | | | | | | |
| EXP-23- | 92 | 0.02 | 99.37 | n.d. | n.d. | n.d. | <100 |
| AA8227 | | | | | | | (60) |
| (Standard | | | | | | | |
| conditions) | | | | | | | |
| EXP-23- | 82 | 0.14 | 99.46 | 0.07 | n.d. | n.d. | 1033 |
| AA8228 | | | | | | | |
| (Standard | | | | | | | |
| conditions) | | | | | | | |
| EXP-23- | 66 | 0.05 | 99.60 | n.d. | <100 | n.d. | <100 |
| AA8229 | | | | | (34) | | (13) |
| (Standard | | | | | | | |
| conditions) | | | | | | | |
| EXP-23- | 76 | 0.02 | 99.64 | n.d. | n.d. | n.d. | 180 |
| AA8230 | | | | | | | |
| (Standard | | | | | | | |
| conditions) | | | | | | | |
| EXP-23- | 89 | n.d. | 99.64 | n.d. | <100 | n.d. | 181 |
| AA8231 | | | | | (7) | | |
| (Standard | | | | | | | |
| conditions) | | | | | | | |
| EXP-23- | 95 | n.d. | 99.66 | n.d. | <100 | n.d. | 621 |
| AA8232 | | | | | (n.d.) | | |
| (Standard | | | | | | | |
| conditions) | | | | | | | |
| EXP-23- | 92 | n.d. | 99.66 | n.d. | <100 | n.d. | <100 |
| AA8233 | | | | | (29) | | (46) |
| (Standard | | | | | | | |
| conditions) | | | | | | | |

After optimizing the crystallization condition, it was possible to obtain material in 50 g scale with very high and consistent purity (equal or above 99% a). As shown in the table above, this technique allows the removal of diesters, tetraesters, as well as MCPD and glycidol (below the detection limit of 100 ppb).

### Example 6: Exemplary Work Flow

Glycerol was obtained from Verbio. Caprylic acid was obtained from Fisher Scientific. Na₂CO₃ was obtained from Thermo Scientific. Concentrated sulfuric acid was obtained from Merck. The chloride content in the reactants was all under <10 ppm or not detectable.

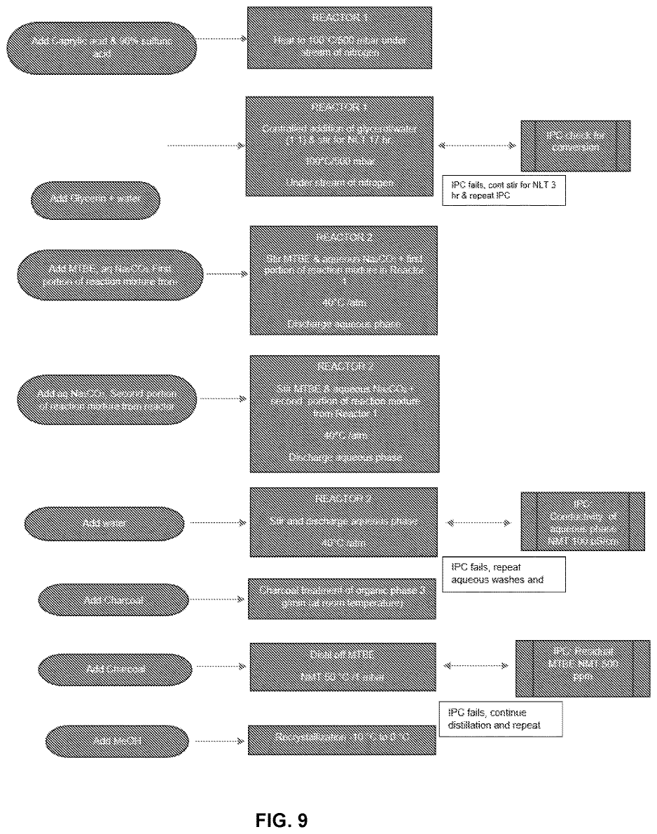

FIG. 9 shows a process to make an ultra-pure composition of tricaprilin. Caprylic acid (99.6% purity) and concentrated sulfuric acid (96%) were added to a first vessel. The vessel was heated to 100° C. and pressurized to 500 mbar under a stream of nitrogen. A 1:1 ratio of glycerol (>99.5%) and water were added to the first vessel and stirred. The vessel was maintained at 100° C. and 500 mbar. In-process samples were taken for the vessel to check for conversion. After conversion was observed, a portion of the reaction mixture from vessel 1 was added to a second vessel. MTBE (>99%) and an aqueous solution of Na₂CO₃ (>99%) were also added to the second vessel and the vessel was heated to 40° C. The pressure of the second vessel was 1 atm. The contents of the second vessel were stirred and the aqueous phase discharged. A second portion of the reaction mixture was vessel 1 was added to vessel 2, MTBE, and an aqueous solution of Na₂CO₃ were added to vessel 2 and the vessel was maintained at 40° C. and 1 atm. The contents of the second vessel were stirred and the aqueous phase discharged. Additional water was added to vessel 2, the vessel was maintained at 40° C. and 1 atm. The aqueous phase discharged. In-process samples were removed from vessel 2 and their conductivity measured. Water added, stirring, and removal of the aqueous phase was continued until the conductivity of the aqueous phase was less than 100 μS/cm. After the desired conductivity was reacted, charcoal was added to the second vessel for charcoal treatment of the remaining organic phase at 3 g/min. The charcoal treatment was performed at room temperature. After the charcoal treatment, MTBE was distilled off by heating the second vessel to 50° C. and 1 mbar pressure. In process samples were tested for MTBE by gas chromatography. The distillation was continued until the amount of residual MTBE was 500 ppm. The remaining product mixture was recrystallized by cooling the vessel to a temperature of between −10° C. to about 0° C. After recrystallization, any residual solvent was removed by heating the vessel to 50° C. and 1 mbar. The resulting product was analyzed by gas chromatography.

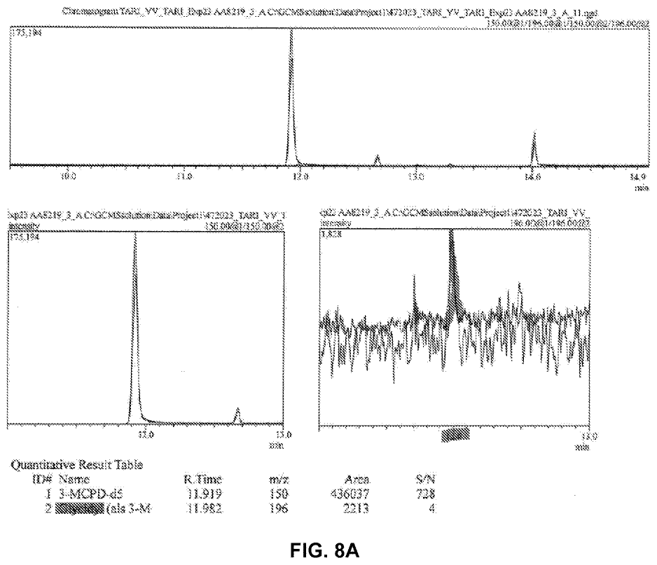

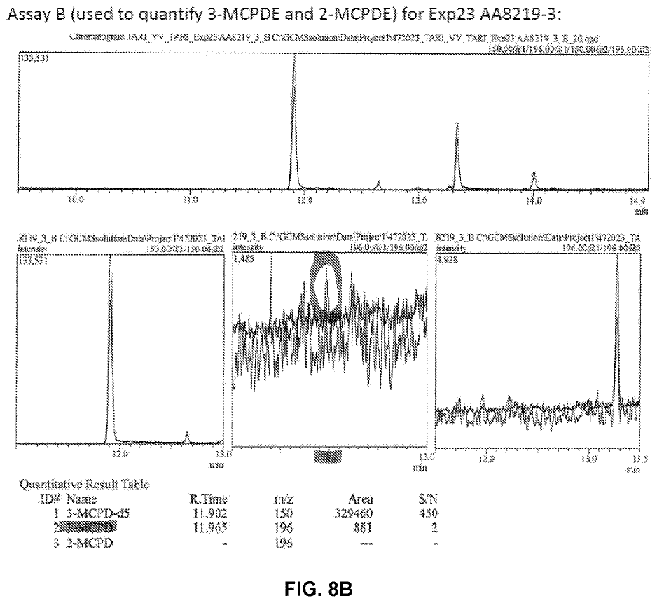

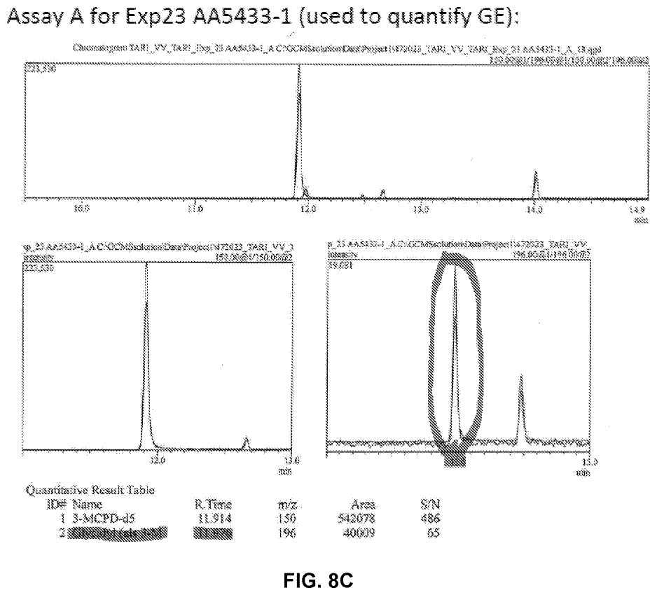

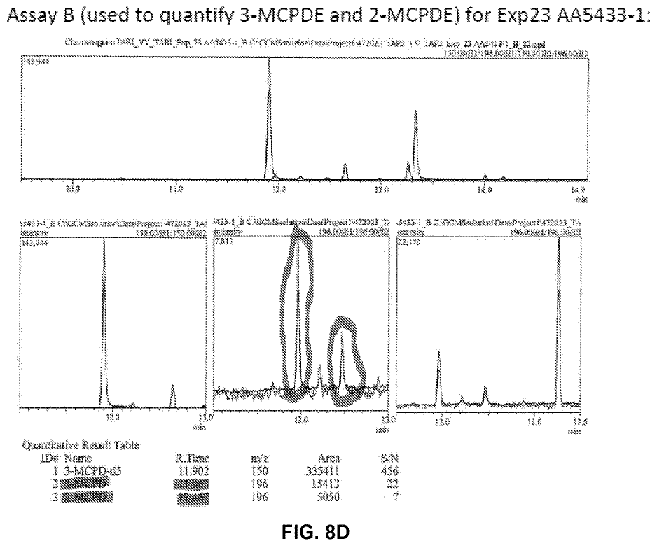

Table 22 shows the results of 6 batches of tricaprilin produced according to the Exemplary procedure. FIGS. 8A-8D provide CG-MS analysis of the products.

TABLE 22
List of experiment according to exemplary procedure

| | | | Sum of | | | 3- | 2- | |
|---|---|---|---|---|---|---|---|---|
| | Yield | Monoesters | Diesters | Tricaprilin | Tetraesters | MCPD | MCPD | Glycidol |
| Batch | [%] | [% a] | [% a] | [% a] | [% a] | [% a] | [% a] | [ppb] |
| EXP-23- | 97.8 | n.d. | 0.36 + 0.75 | 97.6 | 0.06 + 0.15 | <100 | <100 | 2236 |
| AA3527 | | | | | | | | |
| (10 g scale) | | | | | | | | |
| Crystallized | 94.2 | n.d. | n.d. | 99.6 | n.d. | <100 | <100 | <100 |
| product | | | | | | | | |
| EXP-23- | | | | | | | | |
| AA8222 | | | | | | | | |
| EXP-23- | 96.1 | 0.01 | 4.9 | 93.7 | 0.06 + 0.15 | <100 | <100 | 2236 |
| AA6336 | | | | | | | | |
| (10 g scale) | | | | | | | | |
| Crystallized | 88.5 | n.d. | n.d. | 99.7 | n.d. | <100 | <100 | <100 |
| product | | | | | | | | |
| EXP-23- | | | | | | | | |
| AA8224 | | | | | | | | |
| EXP-23- | 96.1 | 0.02 | 13.4 | 84.1 | 0.05 | 217 | <100 | 3449 |
| AA6338 | | | | | | | (71) | |
| (10 g scale) | | | | | | | | |
| Crystallized | 76.2 | tba | tba | tba | tba | <100 | <100 | 180 |
| product | | | | | | (n.d.) | (n.d.) | |
| EXP-23- | | | | | | | | |
| AA8230 | | | | | | | | |
| EXP-23- | 81.1 | 0.2 | 27.8 | 70.8 | 0.06 | <100 | <100 | 2236 |
| AA6339 | | | | | | | | |
| (20 g scale) | | | | | | | | |
| Crystallized | 66 | n.d. | 0.05 | 99.6 | n.d. | <100 | <100 | <100 |
| product | | | | | | | | |
| EXP-23- | | | | | | | | |
| AA8229 | | | | | | | | |
| EXP-23- | 97 | 0.02 | 2.07 | 96.66 | 0.08 + 0.03 | 718 | 198 | 1099 |
| AA6340 | | | | | | | | |
| (20 g scale) | | | | | | | | |
| Crystallized | 92.5 | n.d. | 0.05 | 99.66 | n.d. | <100 | <100 | <100 |
| product | | | | | | | | |
| EXP-23- | | | | | | | | |
| AA8233 | | | | | | | | |
| EXP-23- | 98 | n.d. | 0.22 | 98.76 | 0.07 + 0.03 | 173 | <100 | 4141 |
| AA6341 | | | | | | | | |
| (10 g scale) | | | | | | | | |
| Crystallized | 94.8 | n.d. | n.d. | 99.66 | n.d. | <100 | <100 | 621 |
| product | | | | | | | | |
| EXP-23- | | | | | | | | |
| AA8232 | | | | | | | | |

What is claimed is:

1. An ultra-pure composition comprising at least about 99.2% by weight tricaprilin and about 0.8% by weight or less of impurities,
wherein the tricaprilin is a tri-ester tricaprilin; and
wherein the impurities comprise one or more of 2-monochloropropanediol, 2-monochloropropanediol monoester, 2-monochloropropanediol diester, 3-monochloropropanediol, 3-monochloropropanediol monoester, 3-monochloropropanediol diester, or any combination thereof.

2. The ultra-pure composition of claim 1, comprising at least about 99.5% by weight tricaprilin and about 0.5% by weight or less of impurities.

3. The ultra-pure composition of claim 1, comprising at least about 99.7% by weight tricaprilin and about 0.3% by weight or less of impurities.

4. The ultra-pure composition of claim 1, wherein 3-monochloropropanediol, 3-monochloropropanediol monoester, and 3-monochloropropanediol diester are present in a concentration of 35 ppb or less.

5. The ultra-pure composition of claim 1, comprising less than 35 ppb of each of 3-monochloropropanediol and 3-monochloropropanediol diester.

6. The ultra-pure composition of claim 1, wherein the impurities further comprise glycidol, mono-ester tricaprilin, di-ester tricaprilin, or tetra-ester tricaprilin.

7. The ultra-pure composition of claim 6, wherein the impurities further comprise less than 35 ppb of glycidol.

8. The ultra-pure composition of claim 1, wherein the impurities further comprise unreacted reactants.

9. A method of making an ultra-pure composition of tricaprilin, the method comprising:
(a) feeding caprylic acid, a concentrated acid, glycerol, and water into a first reactor;
(b) reacting caprylic acid, the concentrated acid, glycerol, and water to form a product mixture comprising tricaprilin and impurities in the first reactor, wherein the impurities comprise unreacted reactants and optionally undesired side products;
(c) adding an organic solvent and an aqueous solution of a basic salt to a second reactor forming a biphasic mixture comprising a top organic phase and a bottom aqueous phase;
(d) adding portions of the product mixture from step (b) into the biphasic mixture in step (c) to remove the unreacted reactants, wherein the unreacted reactants are in the aqueous phase;
(e) removing the bottom aqueous phase from the second reactor;
(f) treating the top organic phase with charcoal;
(g) distilling off the organic solvent and leaving tricaprilin and impurities in the second reactor; and
(h) recrystallizing the tricaprilin and impurities to remove the impurities and form the ultra-pure tricaprilin.

10. The method of claim 9, wherein step (a) further comprises:
(ai) co-feeding caprylic acid and concentrated acid into the first reactor to form a mixture;
(aii) heating the mixture to a first temperature under an inert atmosphere; and
(aiii) adding Glycerol and water to the mixture over a first specified time.

11. The method of any one of claims 9-10, wherein step (b) further comprises:
(bi) stirring the product mixture for a second specified time at a second temperature;
(bii) removing a first in process sample from the first reactor;
(biii) checking the first in process sample for conversion, wherein conversion is reaction of hydroxyl groups in the Glycerol to ester groups in tricaprilin; and
(biv) repeating step (bii) to step (biii) until conversion occurs.

12. The method of any one of claims 9-11, wherein step (g) further comprises
(gi) removing a second in process sample from the second reactor;
(gii) measuring amount of the organic solvent remaining in the tricaprilin and impurities; and
(giii) repeating steps (gi) to (gii) until the amount of the organic solvent remaining is 500 ppm or less.

13. The method of any one of claims 9-12, wherein step (h) further comprises
(hi) adding a polar solvent to the tricaprilin and impurities forming a second mixture comprising a polar solvent, tricaprilin, and impurities;
(hii) cooling the second mixture to a third temperature;
(hiii) adding seeding crystals to the second mixture forming a third mixture comprising seeding crystals and the second mixture;
(hiv) stirring the third mixture for a third specified time at a third temperature;
(hv) cooling the third mixture to a fourth temperature in a fourth specified time;
(hvi) maintaining the third mixture at the fourth temperature for a fifth specified time;
(hvii) adding water to the third mixture;
(hviii) maintaining the third mixture at the fourth temperature;
(hix) filtering the third mixture on a filter cooled at the fourth temperature;
(hx) washing the third mixture with an aqueous solution of the polar solvent at a fifth temperature;
(hxi) repeating the washing step (hx);
(hxii) collecting crystals of tricaprilin; and
(hxiii) evaporating residual solvent from the crystals using a rotary evaporator.

14. The method of claim 9, wherein the concentrated acid comprises sulfuric acid.

15. The method of claim 10, wherein the first temperature ranges from about 90° C. to about 120° C.

16. The method of claim 10, wherein the first specified time ranges from about 3 hours to about 5 hours.

17. The method of claim 11, wherein the second temperature ranges from about 90° C. to about 120° C.

18. The method of claim 11, wherein the second specified time ranges from about 15 hours to about 24 hours.

19. A pharmaceutical composition comprising the ultra-pure composition of claim 1.

20. A method of prophylactically or therapeutically treating a disease or a disorder in a subject in need thereof, the method comprising administering a composition comprising ultra-pure tricaprilin to the subject.

21. The method of claim 20, wherein the disease or disorder is any one or more of craniocerebral trauma, stroke, hypoxia, myocardial infarction, refeeding syndrome, anorexia, epilepsy, neurodegenerative diseases such as dementia, Alzheimer's disease, Parkinson's disease, multiple sclerosis and amyotrophic lateral sclerosis, fat metabolic diseases such as glucose transporter defect (GLUT1 defect), VL-FAOD and mitochondriopathies such as mitochondrial thiolase defect, Huntington's disease, cancers such as T-cell lymphomas, astrocytoma and glioblastoma, HIV, rheumatic diseases such as rheumatoid arthritis and arthritis urica, chronic inflammatory bowel diseases, ulcerative colitis, Crohn's disease, lysosomal storage diseases, sphingolipidosis, especially Niemann-Pick disease, diabetes mellitus, migraine and migraine headaches, epilepsy, especially infantile spasms as well as effects or side-effects of chemotherapy.

22. The method of claim 20, wherein the method comprises administering the ultra-pure composition of tricaprilin of claim 1 to the subject.

23. The method of claim 20, wherein the method comprises administering ultra-pure composition of tricaprilin prepared by the method of claim 7 to the subject.

24. The method of claim 20, wherein the ultra-pure composition of tricaprilin is administered orally.
